(19) United States
(12) Patent Application Publication
(10) Pub. No.: US 2026/0139225 A1
(43) Pub. Date: May 21, 2026

(54) GRID-FREE METHODS FOR MAKING GAMMA DELTA T CELLS

(71) Applicant: TAKEDA PHARMACEUTICAL COMPANY LIMITED, Osaka (JP)

(72) Inventors: Yamuna RAGHVANI, London (GB); Shristi BHANDARI, London (GB); Samantha Campo-Redondo HARTMANN, London (GB); Ines SA PEREIRA, London (GB); Puja KAPOOR, London (GB); Przemyslaw KRUCZEK, London (GB); Peter MITCHELL, London (GB); Andrew HUTTON, London (GB)

(21) Appl. No.: 19/447,221

(22) Filed: Jan. 13, 2026

Related U.S. Application Data
(63) Continuation of application No. PCT/IB2024/057036, filed on Jul. 19, 2024.
(60) Provisional application No. 63/527,994, filed on Jul. 20, 2023.

(51) Int. Cl. C12N 5/0783; A61K 35/17; A61K 40/11; A61K 40/31; A61K 40/42; C12N 5/00

(52) U.S. Cl. CPC C12N 5/0636; A61K 35/17; A61K 40/11; A61K 40/31; A61K 40/42; C12N 5/0087; C12N 5/522; C12N 2500/32; C12N 2500/90; C12N 2501/2302; C12N 2501/2304; C12N 2501/2307; C12N 2501/2315; C12N 2501/2321; C12N 2501/998

(57) ABSTRACT

The present disclosure provides, among other things, a method of isolating and expanding gamma delta (γδ) T cells, wherein the method comprises: (a) isolating non-hematopoeitic tissue by biopsy or explant, and (b) culturing the isolated non-hematopoeitic tissue in the absence of a three-dimensional scaffold or grid, thereby expanding and isolating gamma delta (γδ) T cells.

### CROSS-REFERENCE TO RELATED APPLICATIONS

This application is a continuation application of International Application No. PCT/IB2024/057036, filed on Jul. 19, 2024, which claims priority to U.S. Provisional Patent Application No. 63/527,994 filed on Jul. 20, 2023, the contents of both of which are herein incorporated by reference in their entireties, for all purposes.

### BACKGROUND

Gamma delta T cells (γδ T cells) are T cells that express a unique T-cell receptor (TCR) composed of one γ-chain and one δ-chain. Gamma delta T cells are of low abundance in the body, are found in the gut mucosa, skin, lungs and uterus, and are involved in the initiation and propagation of immune responses.

Gamma delta T cells resident in normal non-hematopoeitic tissue (e.g., skin) likely conduct immunosurveillance. Existing techniques allow isolation of only few cells by mechanical dissociation combined with chelating agents or collagenase. Alternately, T cell clones are used.

Additional methods include culturing explants of non-hematopoeitic tissue (e.g., skin) on three-dimensional matrices, e.g., a tantalum coated carbon matrix, leading to the outgrowth of dermal fibroblasts that elaborate T cell chemoattractant factors, which lead to the migration of skin resident T cells out of skin explants where they can be isolated. Tantalum grids are composed of porous, reticulated three-dimensional carbon structure coated with tantulum as an inert, biocompatible scaffold capable of promoting lymphocyte isolation/growth. However, non-seal coated grids pose challenges since they are prone to contamination with tantalum and carbon particulates in culture and are not suitable for GMP manufacturing on a large scale. Further, such particulates pose a safety risk in drug product.

There is a need for efficient and cost-effective methods of isolating gamma delta T cells of high quality on a large scale that is safe for therapeutic use, without associated problems of contamination.

### SUMMARY OF THE INVENTION

The present disclosure provides, among other things, methods of isolating and expanding gamma delta (γδ) T cells, from non-hematopoietic tissue (e.g., skin tissue or gut tissue) in the absence of a three-dimensional scaffold or grid, suitable for expanding and isolating gamma delta (γδ) T cells for therapeutic use. The inventors have carefully selected and identified a new growth cocktail comprising a mixture of cytokines for enhanced composition of gamma delta T cells, better expansion and lower alpha beta (αβ) content. The cytokine mixtures of the present disclosure were selected to provide comparable viability, total cell yield and extended phenotype, i.e., comparable functional potency, between grid and grid-free isolations. Without being bound by theory, gamma delta (γδ) T cell compositions isolated and expanded by grid-free methods described herein are believed to be free of contaminating particles in the final drug product and increase ease of manufacturing to comparable yields and functional characteristics as cells isolated from grid cultures, without requiring additional steps to filter or remove such contaminants, thus reducing associated manufacturing costs. Gamma delta (γδ) T cell compositions isolated and expanded by grid-free methods described herein are used for a variety of ex vivo therapeutic uses, including treating cancer, for example, acute myeloid leukemia, infectious diseases, or inflammatory diseases.

In some aspects, provided herein is a method of isolating and expanding gamma delta (γδ) T cells, wherein the method comprises: (a) isolating non-hematopoeitic tissue by biopsy or explant, and (b) culturing the isolated non-hematopoeitic tissue in the absence of a three-dimensional scaffold or grid, thereby isolating and expanding gamma delta (γδ) T cells.

In some aspects, provided herein is a method of isolating and expanding gamma delta (γδ) T cells, wherein the method comprises: (a) isolating non-hematopoeitic tissue by biopsy or explant, and (b) culturing the isolated non-hematopoeitic tissue in the presence of one or more cytokines selected from the group consisting of IL-2, IL-15, IL-4, IL-7, IL-21 and combinations thereof; and in the absence of a three-dimensional scaffold or grid, thereby isolating and expanding gamma delta (γδ) T cells.

In some embodiments, the gamma delta (γδ) cells are Vdelta1+ (Vδ1+) cells.

In some embodiments, the non-hematopoeitic tissue is treated with the one or more cytokines after initiation of culturing.

In some embodiments, the non-hematopoeitic tissue is treated with the one or more cytokines weekly for the duration of the culturing.

In some embodiments, the non-hematopoeitic tissue is treated with one or more cytokines at least on the day of isolating non-hematopoeitic tissue (D0), after 7 days of culturing (D7) and after an additional 7 days of culturing (D14).

In some embodiments, 6 days after isolation of non-hematopoietic tissue, cells expressing αβ T cell receptor are removed.

In some embodiments, the non-hematopoeitic tissue is skin tissue. In some embodiments, the non-hematopoeitic tissue is gut tissue.

In some embodiments, the removal of cells expressing αβ T cell receptor is carried out using magnetic-activated cell sorting (MACS).

In some embodiments, the MACS is carried out using QuadraMACS or CliniMACS. In some embodiments, the MACS is carried out using QuadraMACS. In some embodiments, the MACS is carried out using CliniMACS.

In some embodiments, the skin tissue comprises between 1×10⁸ to 2×10¹² T cells per 100 cm² surface area. In some embodiments, the skin tissue comprises 1×10⁸ T cells per 100 cm² surface area. In some embodiments, the skin tissue comprises 2×10⁸ T cells per 100 cm² surface area. In some embodiments, the skin tissue comprises 5×10⁸ T cells per 100 cm² surface area. In some embodiments, the skin tissue comprises 1×10⁹ T cells per 100 cm² surface area. In some embodiments, the skin tissue comprises 2×10⁹ T cells per 100 cm² surface area. In some embodiments, the skin tissue comprises 5×10⁹ T cells per 100 cm² surface area. In some embodiments, the skin tissue comprises 1×10¹⁰ T cells per 100 cm² surface area. In some embodiments, the skin tissue comprises 2×10¹⁰ T cells per 100 cm² surface area. In some embodiments, the skin tissue comprises 5×10¹⁰ T cells per 100 cm² surface area. In some embodiments, the skin tissue comprises 1×10¹¹ T cells per 100 cm² surface area. In some embodiments, the skin tissue comprises 2×10¹¹ T cells per 100 cm² surface area. In some embodiments, the skin tissue comprises 5×10¹¹ T cells per 100 cm² surface area. In some embodiments, the skin tissue comprises 1×10¹² T cells per 100 cm² surface area. In some embodiments, the skin tissue comprises 2×10¹² T cells per 100 cm² surface area. In some embodiments, the skin tissue comprises 5×10¹² T cells per 100 cm² surface area.

In some embodiments, the cells are cultured in a GREX100M unit.

In some embodiments, the cells are cultured in a serum-free medium formulated for growing T cells comprising glutamine.

In some embodiments, the medium is an AIM V medium.

In some embodiments, cells are cultured in a medium formulated for growing T cells comprising human albumin and glutamine but lacking animal-derived components and antibiotics.

In some embodiments, the medium is a TexMACS Medium.

In some embodiments, the medium is supplemented with one or more cytokines.

In some embodiments, the one or more cytokines is IL-2, IL-15, IL-4 or IL-21.

In some embodiments, IL-4 and IL-21 are added once during culturing.

In some embodiments, IL-4 and IL-21 are added on the day of isolating non-hematopoeitic tissue (D0).

In some embodiments, the one or more cytokines is IL-7, IL-15, IL-4 or IL-21. In some embodiments, IL-4 and IL-7 are added once during culturing.

In some embodiments, IL-4 and IL-7 are added on the day of isolating non-hematopoeitic tissue (D0).

In some embodiments, the one or more cytokines is IL-15 and IL-21.

In some embodiments, the one or more cytokines is IL-15 and IL-2.

In some embodiments, the one or more cytokines is IL-15, IL-21 and IL-7.

In some embodiments, the one or more cytokines is IL-15, IL-21 and IL-4.

In some embodiments, cells are isolated 14 days (D14), 19 days (D19) or 21 days (D21) after initiation of culturing.

In some embodiments, the cells are isolated two weeks after initiation of culturing (D14).

In some embodiments, the isolated cells comprise greater than 1% of γδ cells.

In some embodiments, the isolated cells comprise 1% to 5% of γδ cells.

In some embodiments, the isolated cells comprise 1% to 10% of γδ cells.

In some embodiments, a proportion of γδ cells in the total isolated cells is determined by Fluorescence Activated Cell Sorting (FACS).

In some embodiments, greater than 60% of isolated γδ cells are Vδ1+ T cells.

In some embodiments, greater than 70% of isolated γδ cells are Vδ1+ T cells.

In some embodiments, greater than 80% of isolated γδ cells are Vδ1+ T cells.

In some embodiments, greater than 90% of isolated γδ cells are Vδ1+ T cells.

In some embodiments, greater than 95% of isolated γδ cells are Vδ1+ T cells.

In some embodiments, greater than 99% of isolated γδ cells are Vδ1+ T cells.

In some embodiments, 100% of isolated γδ cells are Vδ1+ T cells.

In some embodiments, the isolated Vδ1+γδ cells have greater than about 80% purity.

In some embodiments, the isolated Vδ1+γδ cells have greater than about 90% purity.

In some embodiments, the isolated Vδ1+γδ cells have greater than about 95% purity.

In some embodiments, the isolated Vδ1+γδ cells have greater than about 99% purity.

In some embodiments, the isolated Vδ1+γδ cells have 100% purity.

In some embodiments, the isolated Vδ1+γδ cells are cryopreserved in a cryoformulation medium.

In some embodiments, the cryoformulation medium comprises CS10.

In some embodiments, the cryoformulation medium comprises TexMACS and CS10.

In some embodiments, the cryoformulation medium comprises TexMACS and CS10 in a 1:1 ratio.

In some embodiments, the isolated cells express one or more SR01 or SR02 markers.

In some embodiments, the isolated cells express one or more SR01 markers selected from CD27, CD45RA, Programmed cell death protein 1 (PD1), T cell immunoreceptor with Ig and ITIM domains (TIGIT), and killer cell lectin-like receptor subfamily D, member 1 (NKG2D). In some embodiments, the isolated cells express CD27. In some embodiments, the isolated cells express CD45RA. In some embodiments, the isolated cells express Programmed cell death protein 1 (PD1). In some embodiments, the isolated cells express T cell immunoreceptor with Ig and ITIM domains (TIGIT). In some embodiments, the isolated cells express killer cell lectin-like receptor subfamily D, member 1 (NKG2D).

In some embodiments, the isolated cells express one or more SR02 markers selected from NKp30, CD56, killer cell lectin-like receptor subfamily A, member 1 (NKG2A), and killer cell lectin-like receptor subfamily C, member 1 (NKG2C). In some embodiments, the isolated cells express NKp30. In some embodiments, the isolated cells express CD56. In some embodiments, the isolated cells express killer cell lectin-like receptor subfamily A, member 1 (NKG2A). In some embodiments, the isolated cells express killer cell lectin-like receptor subfamily C, member 1 (NKG2C).

In some embodiments, the isolated cells comprise 60% or greater CD27 cells.

In some embodiments, the isolated cells comprise greater than 85% CD45+ cells.

In some embodiments, the isolated cells comprise greater than 90% CD45+ cells.

In some embodiments, the isolated cells comprise greater than 95% CD45+ cells. In some embodiments, the isolated cells comprise greater than 99% CD45+ cells.

In some embodiments, the isolated cells comprise 100% CD45+ cells.

In some embodiments, the isolated cells comprise less than 80% CD39+ cells.

In some embodiments, the isolated cells comprise greater than 80% CD227+ cells. In some embodiments, the isolated cells comprise 10% or less Vδ2+ cells.

In some embodiments, the isolated cells have a viability of 70% or greater.

In some embodiments, the isolated cells have a viability of 80% or greater.

In some embodiments, the isolated cells have a viability of 90% or greater.

In some embodiments, the isolated cells have a viability of 95% or greater.

In some embodiments, the isolated cells have a viability of 99% or greater.

In some embodiments, the isolated cells have a viability of 100%.

In some embodiments, the isolated cells have a yield of between about 1.5×10⁹ to about 2.5×10⁹ cells per 100 cm² surface area.

In some embodiments, the isolated cells have a yield of about 1.5×10⁹ cells per 100 cm² surface area.

In some embodiments, the isolated cells have a yield of about 2×10⁹ cells per 100 cm² surface area.

In some embodiments, the isolated cells have a yield of about 2.5×10⁹ cells per 100 cm² surface area.

In some embodiments, the isolated cells are free of grid impurities.

In some embodiments, the isolated cells are free of grid impurities selected from polymers, ceramics, or metals.

In some embodiments, the isolated cells are free of impurities selected from tantalum or carbon particulates.

In some embodiments, the cells do not express a chimeric antigen receptor (CAR).

In some embodiments, the cells are transduced to express a chimeric antigen receptor (CAR).

In some embodiments, provided herein is a composition comprising gamma delta (γδ) T cells manufactured by a method described herein.

In some embodiments, provided herein is a method of treating a disease, the method comprising administering a therapeutically effective amount of the composition described herein to a subject in need thereof.

In some embodiments, the disease is a cancer, an infectious disease or an inflammatory disease.

Any numerals used in this application with or without the terms “about” or “approximately” are meant to cover any normal fluctuations appreciated by one of ordinary skill in the relevant art.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### BRIEF DESCRIPTION OF THE DRAWINGS

The drawings are for illustration purposes and are in no way limiting.

FIG. 1 is a graph that shows viability of gamma delta (γδ) cells from exemplary grid or grid-free isolations is comparable at about 85%.

FIG. 2A is a graph that shows percentage of gamma delta (γδ) cells from exemplary grid or grid-free isolations is comparable, though slightly lower in grid-free isolation at about 4% relative to about 6% from grid isolation.

FIG. 2B is a graph that shows γδ yield is slightly lower in exemplary grid-free isolation, but most donors meet minimum γδ% specification (>1%).

FIG. 3 is a graph that shows the proportion of CD27 cells in three exemplary grid-free cell preparations ranging from 30% to 70%. In version 3, the CD27 percentage was about 70%.

FIG. 4A is a graph of Vδ1 CD227 vs. percentage of γδ CD39 cells. Based on the graph, high-expanding donors were selected. FIG. 4B is graph of percentage of total cell fold increase on D14 relative to percentage of Vδ1 CD227 cells. FIG. 4C is a graph of percentage of total cell fold increase on D14 relative to percentage of γδ CD39 cells.

FIG. 5A depicts a graph showing viability of the cells was comparable between various cytokine combinations tested. FIG. 5B is a graph of total cell numbers between various cytokine combinations depicting that yield of the cells was comparable to GEN2 in combinations 1-4, but decreased significantly in combinations 5 and 6, while yield increased in at least 3 of 4 donors in combinations 7-10. FIG. 5C depicts a graph of percentage of gamma delta (γδ) T cells relative to total live cells showing that it was comparable in different cytokine combinations, if not higher than control GEN2. FIG. 5D is a graph that showed that the average percentage of V-delta 1 type of gamma delta T cells was comparable, or higher than GEN2 for cytokine combinations 5-10. FIG. 5E is a graph that showed that cytokine combinations 3 and 4 have a higher percentage on average of non-V-delta 1 type of gamma delta T cells than GEN2. FIG. 5F is a graph that showed that cytokine conditions 2, 3, 7, 8, 9 and 10 showed higher γδ cell number yields than GEN2. FIG. 5G is a graph that showed that cytokine combinations 2, 3, 7, 8, 9 and 10 showed higher numbers of Vδ1 cells. FIG. 5H is a graph that showed that cytokine conditions 5 and 6 showed a lower percentage of non-V-delta 1 type cells. FIG. 5I is a graph that showed that the percentage of alpha beta cells was slightly higher than GEN2 in cytokine conditions 5-10 in at least 3 out of 4 donors. FIG. 5J is a graph that showed that the percentage of DN cells was also reduced when compared to GEN2. FIG. 5K is a graph that showed the percentage of CD27 positive V delta 1 cells. The results showed that CD27 expression was higher than GEN2 in cytokine combinations 5-10. FIG. 5L showed the percentage of PD1 positive V delta 1 cells was higher than GEN2 for cytokine conditions 3, 5 and 6. FIG. 5M showed that the percentage of TIGIT positive V delta 1 cells was comparable across conditions except for lower expression for cytokine condition 3. FIG. 5N is a graph that showed the percentage of NKG2D positive V delta 1 cells was comparable across conditions except for lower expression for condition 3.

FIG. 6A-FIG. 6D showed that conditions 5-10 have enhanced naïve cell phenotypes (CD27 (+) CD45RA (+)) as measured by the presence of both CD27 and CD45R cell surface markers. FIG. 6A depicts terminally differentiated effector memory (CD27(−) CD45RA (+)) population. FIG. 6B depicts naïve cell (CD27 (+) CD45RA (+)) population. FIG. 6C depicts effector memory (CD27(−) CD45RA(−)) population. FIG. 6D depicts central memory (CD27 (+) CD45RA(−)) phenotypes.

FIG. 7A-FIG. 7D showed SR02 marker expression. FIG. 7A is a graph that showed NKp30 expression was comparable to GEN2. FIG. 7B is a graph that showed that NKG2A expression increased relative to GEN2 for cytokine conditions 5-10, with the highest increase seen for cytokine conditions 5 and 6. FIG. 7C is a graph that showed NKG2C expression is comparable to GEN2. FIG. 7D is a graph that showed CD56 expression increases relative to GEN2 for at least 3 of 4 donors for conditions 5-10.

FIG. 8A-FIG. 8D showed NKG2C and NKG2A cell surface markers. FIG. 8A is a graph that showed proportion of NKG2C-NKG2A+ cells. FIG. 8B is a graph that showed proportion of NKG2C+NKG2A+ cells. FIG. 8C is a graph that showed proportion of NKG2C-NKG2A-cells. FIG. 8D is a graph that showed proportion of NKG2C+NKG2A-cells.

### DEFINITIONS

In order for the present disclosure to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term refers to a range of values that fall within 10% of the stated reference value. In some embodiments, the term refers to a range of values that fall within 5% of the stated reference value. The term “between” includes the values of the specified boundaries and all intervening values and fractions.

Allogeneic: As used herein, allogeneic refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

Autologous: As used herein, the term “autologous” means from the same individual. For example, “autologous” in relation to donor and recipient means that the donor subject is the recipient subject.

Bioreactor: As used herein, the term “bioreactor” refers to a large-scale cell culture system that provides nutrients to cells and removes metabolites, as well as furnishes a physio-chemical environment conducive to cell growth, in a closed sterile system. In particular aspects, the biological and/or biochemical processes develop under monitored and controlled environmental and operating conditions, for example, pH, temperature, pressure, nutrient supply and waste removal. According to the present disclosure, the basic class of bioreactors suitable for use with the present methods includes hollow fiber bioreactors.

Closed System: The term “closed system” refers to a system sealed to ensure fluid sterility either by hermetically sealing the entire system or by providing sterile barrier filters at all connections to the collection system.

Cryopreservation: As used herein, the term “cryopreservation” generally refers to a freezing a biological material (e.g., a population of cells) to low enough temperatures, such that chemical processes, which might otherwise damage the material are halted thereby preserving the material. Cryopreserved cells maintain viability for an extended period of time in the frozen state, such as for 1, 5, 10 or more years in the cryopreserved state. The cryopreserved cells, once thawed, are able to propagate both for in vitro and in vivo applications.

Cryoprotectant: As used herein, the term “cryoprotectant” means a substance used to protect biological tissue from freezing damage. Exemplary cryoprotectants include, for example, dimethyl sulfoxide (DMSO), glycerol, ethylene glycol and propanediol.

Cancer: As used herein, “cancer” refers to the abnormal proliferation of malignant cancer cells and includes leukemias, such as acute myeloid leukemia (AML), chronic myeloid leukemia (CML), acute lymphoblastic leukemia (ALL) and chronic lymphocytic leukemia (CLL), lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer. Cancer cells within cancer patient may be immunologically distinct from normal somatic cells in the individual (e.g., the cancerous tumor may be immunogenic). For example, the cancer cells may be capable of eliciting a systemic immune response in the cancer patient against one or more antigens expressed by the cancer cells. The antigens that elicit the immune response may be tumor antigens or may be shared by normal cells. A patient with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine (Longo D L, Fauci A S, Kasper D L, Hauser S L, Jameson J, Loscalzo J. eds. 18e. New York, NY: McGraw-Hill; 2012). In some instances, a diagnosis of a cancer in an individual may include identification of a particular cell type (e.g., a cancer cell) in a sample of a body fluid or tissue obtained from the individual.

Expanded population of Gamma Delta T cells: As used herein, “expanded” or “expanded population of gamma delta T cells” includes populations of cells which are larger or contain a larger number of cells than a non-expanded population. Such populations may be large in number, small in number or a mixed population with the expansion of a proportion or particular cell type within the population. It will be appreciated that the term “expansion step” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g., fold-increase or fold-expansion) are illustrative of an increase in the number or size of a population of cells or the number of cells and are indicative of the amount of expansion.

Ex vivo: As used herein, the term “ex vivo” means a process in which cells are removed from a living organism and are propagated outside the organism (e.g., in a test tube, in a culture bag, in a bioreactor).

Enriched population of Gamma Delta T cells: As used herein, enriched population refers to an allogeneic composition of gamma delta cells depleted of αβ cells.

Fresh cell or Rescued Fresh Cell: As used herein, the terms “fresh,” “fresh cell,” or “rescued fresh cell” refers to mammalian cells that have never been frozen and/or once frozen but subsequently restimulated, cultured in culture medium and then harvested as fresh cells.

Functional equivalent or derivative: As used herein, the term “functional equivalent” or “functional derivative” denotes, in the context of a functional derivative of an amino acid sequence or any other molecule (e.g., a media formulation component) that retains an activity (either function or structural) that is substantially similar to that of the original molecule or sequence. A functional derivative or equivalent may be a natural derivative or is prepared synthetically. Exemplary derivatives include those having chemico-physical properties which are similar to that of the original molecule or sequence. Desirable similar chemico-physical properties include similarities in charge, bulkiness, hydrophobicity, hydrophilicity, and the like.

Isotonic: As used herein, the term “isotonic” means having an osmotic pressure that is equal to or approximately the same as the osmotic pressure of a physiological fluid.

Isolating cells: As used herein, “isolating” or “isolation of cells”, for example, of lymphocytes and/or gamma delta T cells, refer to methods or processes wherein cells are removed, separated, purified, enriched or otherwise taken out from a tissue or a pool of cells. It will be appreciated that such references include the terms “separated”, “removed”, “purified”, “enriched” and the like. Isolation of gamma delta T cells includes the isolation or separation of cells from an intact non-hematopoietic tissue sample or from the stromal cells of the non-hematopoietic tissue (e.g., fibroblasts or epithelial cells). Such isolation may alternatively or additionally comprise the isolation or separation of gamma delta T cells from other hematopoietic cells (e.g., alpha beta T cells or other lymphocytes). Isolation may be for a defined period of time, for example starting from the time the tissue explant or biopsy is placed in the isolation culture and ending when the cells are collected from culture, such as by centrifugation or other means for transferring the isolated cell population to expansion culture or used for other purposes, or the original tissue explant or biopsy is removed from the culture. The isolation step may be for at least about three days to about 45 days. In one embodiment, the isolation step is for at least about 10 days to at least 28 days. In a further embodiment, the isolation step is for at least 14 days to at least 21 days. The isolation step may therefore be for at least three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, about 35 days, about 40 days, or about 45 days. In one embodiment, the isolation step is for 19 days. In a yet further embodiment, the isolation step is for 21 days. It can be appreciated that although isolated cell proliferation may not be substantial during this isolation step, it is not necessarily absent. Indeed, for someone skilled in the art it is recognized that isolated cells may also start to divide to generate a plurality of such cells within the isolation vessel containing the tissue and/or scaffold. Thus, references herein to “isolated gamma delta T cells”, “isolated gamma delta T cell population”, “isolated population of gamma delta T cells”, “separated gamma delta T cells”, “separated gamma delta T cell population” or “separated population of gamma delta T cells” will be appreciated to refer to hematopoietic cells or a population of hematopoietic cells including gamma delta cells that have been isolated, separated, removed, purified or enriched from a non-hematopoietic tissue sample of origin such that the cells are out of substantial contact with non-hematopoietic cells or cells contained within the intact non-hematopoietic tissue. Likewise, references herein to an “isolated or separated population of Vδ1 T cells” refer to hematopoietic cells including Vδ1 T cells that have been isolated, separated, removed, purified or enriched from non-hematopoietic tissue sample of origin such that the cells are out of substantial contact with non-hematopoietic cells or cells contained within the intact non-hematopoietic tissue. Therefore, isolation or separation refers to the isolation, separation, removal, purification or enrichment of hematopoietic cells (e.g., gamma delta T cells or other lymphocytes) from non-hematopoietic cells (e.g., stromal cells, fibroblasts and/or epithelial cells).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Interleukin-4-like activity: As used herein, a growth factor having “interleukin-4-like activity” means any compound that has the same activity as IL-4 with respect to its ability to promote similar physiological effects on γδ T cells in culture and includes, but is not limited to, IL-4 and IL-4 mimetics, or any functional equivalent of IL-4. The physiological effects promoted by IL-4 on γδ T cells have been shown to include the decrease of NKG2D and NCR expression levels, the inhibition of cytotoxic function and improved selective survival. IL-4 has also been shown to significantly inhibit the secretion of pro-inflammatory cytokines, including IFN-γ, TNF-α, from activated TCRγδ+ T cells.

Interleukin-15-like activity: As used herein, a growth factor having “interleukin-15-like activity” means any compound that has the same activity as IL-15 with respect to its ability to promote similar physiological effects on γδ T cells in culture and includes, but is not limited to, IL-15 and IL-15 mimetics, or any functional equivalent of IL-15, including IL-2 and IL-7. The physiological effects promoted by IL-15, IL-2 and IL-7 on cultured γδ T cells include the induction of cell differentiation towards a more cytotoxic phenotype, such as the upregulation of NKG2D and natural cytotoxicity receptors (NCR) (NKp30 and NKp44) expression levels, increased anti-tumor cytotoxic function and increased production of pro-inflammatory cytokines, such as IFN-γ.

Non-hematopoeitic tissue: As used herein, the terms “non-haematopoietic tissues” or “non-hematopoietic tissue sample” include skin (e.g., human skin) and gut (e.g., human gut). Non-hematopoietic tissue is a tissue other than blood, bone marrow, or thymus tissue. In one embodiment, the non-hematopoietic tissue sample is skin (e.g., human skin). In a further embodiment, the non-hematopoietic tissue sample is gut or gastrointestinal tract (e.g., human gut or human gastrointestinal tract). In some embodiments, the lymphocytes and/or gamma delta T cells are not obtained from particular types of samples of biological fluids, such as blood or synovial fluid. In some embodiments, the non-hematopoietic tissue sample from which the lymphocytes and/or gamma delta T cells are isolated according to the methods defined herein is skin (e.g. human skin), which can be obtained by methods known in the art. Alternatively, the methods of isolation of lymphocytes and/or gamma delta T cells provided herein can be applied to the gastrointestinal tract (e.g. colon or gut), mammary gland, lung, prostate, liver, spleen, pancreas, uterus, vagina and other cutaneous, mucosal or serous membranes. The lymphocytes and/or gamma delta T cells may also be resident in human cancer tissue samples, e.g., tumors of the breast or prostate. In some embodiments, the lymphocytes and/or gamma delta T cells may be from human cancer tissue samples (e.g., solid tumor tissues). In other embodiments, the lymphocytes and/or gamma delta T cells may be from non-hematopoietic tissue sample other than human cancer tissue (e.g., a tissue without a substantial number of tumor cells). For example, the lymphocytes and/or gamma delta T cells may be from a region of skin (e.g., healthy skin) separate from a nearby or adjacent cancer tissue. Thus, in some embodiments, the gamma delta T cells are not obtained from human cancer tissue. In further embodiments, the lymphocytes are not obtained from a human cancer tissue. In one embodiment the non-hematopoietic tissue sample of the methods defined herein has been obtained from a human.

Pharmaceutically acceptable carrier: As used herein, pharmaceutically acceptable carrier includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Primary Cell: The term, “primary cell,” refers to cells that are directly isolated from a subject and which are subsequently propagated.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Grid or Scaffold: As used herein, a “grid” or “scaffold” or “synthetic scaffold” refers to a non-native three-dimensional matrix suitable for supporting cell growth. An explant or tissue biopsy may be adhered to a grid to facilitate lymphocyte egress from the explant onto the grid. Grids may be constructed from natural and/or synthetic materials such as polymers (e.g., natural or synthetic polymers, e.g., polyvinyl pyrolidones, polymethylmethacrylate, methyl cellulose, polystyrene, polypropylene, polyurethane), ceramics (e.g., tricalcium phosphate, calcium aluminate, calcium hydroxyapatite), or metals (tantalum, titanium, platinum and metals in the same element group as platinum, niobium, hafnium, tungsten, and combinations of alloys thereof). Biological factors, e.g., collagens (e.g., collagen I or collagen II), fibronectins, laminins, integrins, angiogenic factors, anti-inflammatory factors, glycosaminoglycans, vitrogens, antibodies and fragments thereof or cytokines (e.g., IL-2 or IL-15, and combinations thereof) may be coated onto the surface of a grid or encapsulated within the grid material to enhance cell adhesion, migration, survival, or proliferation, according to methods known in the art. A grid culture is used to isolate tissue-specific lymphocytes, e.g., from non-hematopoietic tissues such as gut, prostate and breast.

Solid tumor: As used herein, a “solid tumor” is any cancer of body tissue other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

SR01 marker: As used herein, SR01 marker includes a panel of markers selected from CD27, CD45RA, Programmed cell death protein 1 (PD1), T cell immunoreceptor with Ig and ITIM domains (TIGIT) and killer cell lectin-like receptor subfamily D, member 1 (NKG2D). In addition, the SR01 marker panel includes lineage markers for pan gamma delta T cells, pan alpha beta T cells and Vdelta1 TCR.

SR02 marker: As used herein, SR02 marker includes a panel of markers selected from NKp30, CD56, killer cell lectin-like receptor subfamily A, member 1 (NKGC2A) and killer cell lectin-like receptor subfamily C, member 1 (NKG2C). In addition, the SR02 marker panel includes lineage markers for pan gamma delta T cells, pan alpha beta T cells and Vdelta1 TCR.

Subject or Patient: As used herein, “subject” or “patient” refers to an individual suffering from a disease or disorder, for example, cancer, solid tumor, infectious disease or inflammatory disease.

Therapeutically effective amount: As used herein, the term “therapeutically effective amount” means an amount effective, at dosages, frequency of administration and for duration of time necessary to achieve the desired results such that one or more symptoms or biomarkers is improved after treatment.

### DETAILED DESCRIPTION

The present disclosure provides, among other things, methods of isolating and expanding gamma delta (γδ) T cells, from skin tissue in the absence of a three-dimensional scaffold or grid, suitable for isolating and expanding gamma delta (γδ) T cells for therapeutic use, by cultivating cells in carefully selected cytokine mixtures providing comparable viability, total cell yield and extended phenotype, i.e., comparable functional potency, between grid and grid-free isolations. The gamma delta (γδ) T cell compositions isolated and expanded by grid-free methods described herein are used for a variety of ex vivo therapeutic uses, including treating cancer, for example, acute myeloid leukemia, infectious diseases, or inflammatory diseases.

In some aspects, provided herein is a method of isolating and expanding gamma delta (γδ) T cells, wherein the method comprises: (a) isolating non-hematopoeitic tissue by biopsy or explant, and (b) culturing the isolated non-hematopoeitic tissue in the presence of one or more cytokines selected from the group consisting of IL-2, IL-15, IL-4, IL-7 and IL-21; and in the absence of a three-dimensional scaffold or grid, thereby isolating and expanding gamma delta (γδ) T cells.

Gamma delta (γδ) T cells

Gamma delta (γδ) T cells are lymphoid cells that undergo maturation in the thymus. Double-negative thymocytes (CD4− CD8−) are differentiated into T cells expressing γδ T cell receptor (TCR). These cells then migrate to peripheral blood (PB) and mucosal tissues, including skin and gut mucosa, where they function as primary effectors in the response against infections and cancer, prior to responses of the αβ T cell lineage.

According to the Lefranc & Rabbits's system nomenclature, four subtypes of human γδ T cells are defined by the TCR 8 chain, namely, Vδ1, Vδ2, Vδ3 and Vδ5 (LeFranc M P et al. (′ell (1986) 45:237-46). Vδ1 and Vδ2 subtypes are the most predominant while Vδ3 cells comprise a major form of the Vδ1-Vδ2-subtype and found in liver, rather than peripheral blood. Vδ5 cells are found in peripheral blood or tissues. Vδ1+γδ T cells, for example, recognize target cells and mediate anti-tumor activity through the direct lysis of transformed cells.

### Grid-Free DermaPack Cultures

One of the methods of isolating gamma delta T cells is culturing explants of non-hematopoeitic tissue (e.g., skin) on three-dimensional matrices, e.g., a tantalum coated carbon matrix, leading to the outgrowth of dermal fibroblasts that elaborate T cell chemoattractant factors, which lead to the migration of skin resident T cells out of skin explants where they can be isolated (Clark et al., 2006). Tantalum grids are composed of porous, reticulated three-dimensional carbon structure coated with tantulum as an inert, biocompatible scaffold capable of promoting lymphocyte isolation/growth. A limitation of three-dimensional grids to cultivate gamma delta T cells, is that tantalum particle contaminants are observed in cell pellets during large scale harvest. Tantalum particles (4-40 μm in size) are also observed in drug substance, as well as the αβ-depleted expansion phase of gamma delta T cells.

The present disclosure provides an efficient and cost-effective method of isolating and expanding gamma delta T cells of high quality from non-hematopoietic tissue (e.g., skin tissue or gut tissue) on a large scale that is safe for therapeutic use.

In some embodiments, the gamma delta (γδ) cells are Vdelta1+ (Vδ1+) cells.

In some aspects, the gamma delta T cell compositions are expanded using exogenous growth factors and have improved polyclonality compared to FACS-sorted, unexpanded gamma delta T cells (i.e. ex vivo gamma delta T cells), therefore in one embodiment, the allogeneic composition comprises gamma delta T cells obtained using an expansion method, in particular, wherein said expansion method comprises culturing gamma delta T cells in the presence of exogenous growth factors.

### Cytokine Treatment

In some embodiments, the non-hematopoeitic tissue is treated with one or more cytokines after initiation of culturing.

In some embodiments, the non-hematopoeitic tissue is treated with one or more cytokines weekly for the duration of the culturing.

In some embodiments, the non-hematopoeitic tissue is treated with one or more cytokines at least on the day of isolating non-hematopoeitic tissue (D0), after 7 days of culturing (D7) and after an additional 7 days of culturing (D14).

In some embodiments, the one or more cytokines are selected from the group consisting of IL-2, IL-15, IL-4, IL-7, IL-21 and combinations thereof.

“In the absence of any given interleukin” refers not only to the complete absence of these cytokines in the culture medium, but also include the use of such cytokines at concentration levels so low that they cannot produce a measurable response or physiological effect in target cells and thus can be considered absent for practical purposes. It should be apparent to any one skilled in the art that cells cultured in the first culture medium must not receive a functionally relevant stimulus by IL-2, IL-7 and IL-15 or functionally similar growth factors. Additionally, cells in the second culture medium must not receive a functionally relevant stimulus by IL-4 or functionally similar growth factors. In some embodiments, these cytokines must not be present in the cell culture medium at a final concentration higher than 2 ng/ml; more preferably, not higher than 1 ng/ml, more preferably not higher than 0.1 ng/ml. In some embodiments, these cytokines are absent.

As used herein, “IL-2” refers to native or recombinant IL-2 or a variant thereof that acts as an agonist for one or more IL-2 receptor (IL-2R) subunits (e.g., mutants, muteins, analogues, subunits, receptor complexes, fragments, isoforms, and peptidomimetics thereof). Such agents can support proliferation of an IL-2-dependent cell line, CTLL-2 (33; American Type Culture Collection (ATCC®) TIB 214).

IL-2 can also refer to IL-2 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-2 variants are also encompassed by the disclosure. Examples of such variants are proteins that result from alternate mRNA splicing events or from proteolytic cleavage of the IL-2 protein, wherein the IL-2 binding property is retained. Alternate splicing of mRNA may yield a truncated but biologically active IL-2 protein. Variations attributable to proteolysis include, for example, differences in the N- or C-termini upon expression in different types of host cells, due to proteolytic removal of one or more terminal amino acids from the IL-2 protein (generally from 1-10 amino acids).

In certain embodiments, the methods defined herein include IL-2 typically at a concentration of at least 10 IU/mL, such as at least 100 IU/mL (e.g., from 10 IU/mL to 1,000 IU/mL, from 20 IU/mL to 800 IU/mL, from 25 IU/mL to 750 IU/L, from 30 IU/mL to 700 IU/mL, from 40 IU/mL to 600 IU/mL, from 50 IU/mL to 500 IU/mL, from 75 IU/mL to 250 IU/mL, or from 100 IU/mL to 200 IU/mL, e.g., from 10 IU/mL to 20 IU/mL, from 20 IU/mL to 30 IU/mL, from 30 IU/mL to 40 IU/mL, from 40 IU/mL to 50 IU/mL, from 50 IU/mL to 75 IU/mL, from 75 IU/mL to 100 IU/mL, from 100 IU/mL to 150 IU/mL, from 150 IU/mL to 200 IU/mL, from 200 IU/mL to 500 IU/mL, or from 500 IU/mL to 1,000 IU/mL). In certain embodiments, the methods defined herein include IL-2 typically at a concentration of less than 1,000 IU/mL, such as less than 500 IU/mL. In some embodiments, the methods include IL-2 at a concentration of about 100 IU/mL. In some embodiments, the methods, such as 138 IU/mL.

In some embodiments, the growth factor having interleukin-15-like activity is either interleukin-15 (IL-15), interleukin-2 (IL-2), or interleukin-7 (IL-7), preferably IL-15. As used herein, “IL-15” refers to native or recombinant IL-15 or a variant thereof that acts as an agonist for one or more IL-15 receptor (IL-15R) subunits (e.g., mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion or substitution. Variants may comprise conservatively substituted sequences, meaning that a given amino acid residue is replaced by a residue having similar physiochemical characteristics. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-15 variants are also encompassed by the disclosure. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids).

In some embodiments, the methods defined herein include IL-15 typically at a concentration of at least 10 IU/mL, such as at least 100 IU/mL, in particular at least 500 IU/mL (e.g., from 10 IU/mL to 1,000 IU/mL, from 20 IU/mL to 900 IU/mL, from 25 IU/mL to 750 IU/mL, from 30 IU/mL to 600 IU/mL, from 40 IU/mL to 500 IU/mL, from 50 IU/mL to 400 IU/mL, from 75 IU/mL to 250 IU/mL, or from 100 IU/mL to 200 IU/mL, e.g., from 100 IU/mL to 900 IU/mL, from 200 IU/mL to 800 IU/mL, from 300 IU/mL to 700 IU/mL, from 400 IU/mL to 600 IU/mL, or from 500 IU/mL to 1,000 IU/mL). In certain embodiments, the methods defined herein include IL-15 typically at a concentration of less than 1,000 IU/mL, such as less than 700 IU/mL. In some embodiments, the methods include IL-15 at a concentration of about 600 IU/mL.

In one embodiment, the growth factor having interleukin-4-like activity is interleukin-4 (IL-4).

As used herein, “IL-4” refers to native or recombinant IL-4 or a variant thereof that acts as an agonist for one or more IL-4 receptor (IL-4R) subunits (e.g., mutants, muteins, analogues, subunits, receptor complexes, fragments, isoforms, and peptidomimetics thereof). Such agents can support differentiation of naive helper T cells (ThO cells) to Th2 cells. Mature human IL-4 occurs as a 129 amino acid sequence (less the signal peptide, consisting of an additional 24 N-terminal amino acids).

IL-4 can also refer to IL-4 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-4 variants are also encompassed by the disclosure. Examples of such variants are proteins that result from alternate mRNA splicing events or from proteolytic cleavage of the IL-4 protein, wherein the IL-4 binding property is retained. Alternate splicing of mRNA may yield a truncated but biologically active IL-4 protein. Variations attributable to proteolysis include, for example, differences in the N- or C-termini upon expression in different types of host cells, due to proteolytic removal of one or more terminal amino acids from the IL-4 protein (generally from 1-10 amino acids).

In some embodiments, the methods defined herein include IL-4 typically at a concentration of at least 1 IU/mL, such as at least 10 IU/mL (e.g., from 1 IU/mL to 1,000 IU/mL, from 5 IU/mL to 500 IU/mL, from 10 IU/mL to 250 IU/mL, from 50 IU/mL to 150 IU/mL, e.g., from 1 IU/mL to 5 IU/mL, from 5 IU/mL to 10 IU/mL, from 10 IU/mL to 50 IU/mL, from 50 IU/mL to 100 IU/mL, from 100 IU/mL to 150 IU/mL). In certain embodiments, the methods defined herein include IL-4 typically at a concentration of less than 500 IU/mL, such as less than 100 IU/mL. In some embodiments, the methods include IL-4 at a concentration of about 100 IU/mL, such as 95 IU/mL.

As used herein, “IL-7” refers to native or recombinant IL-7 or a variant thereof that acts as an agonist for one or more IL-7 receptor (IL-7R) subunits (e.g., mutants, muteins, analogues, subunits, receptor complexes, fragments, isoforms, and peptidomimetics thereof). Mature human IL-7 occurs as a 152 amino acid sequence (less the signal peptide, consisting of an additional 25 N-terminal amino acids).

IL-7 can also refer to IL-7 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-7 variants are also encompassed by the disclosure. Examples of such variants are proteins that result from alternate mRNA splicing events or from proteolytic cleavage of the IL-7 protein, wherein the IL-7 binding property is retained. Alternate splicing of mRNA may yield a truncated but biologically active IL-7 protein. Variations attributable to proteolysis include, for example, differences in the N- or C-termini upon expression in different types of host cells, due to proteolytic removal of one or more terminal amino acids from the IL-7 protein (generally from 1-10 amino acids).

In some embodiments, the methods defined herein include IL-7 typically at a concentration of at least 0.1 ng/mL, such as at least 10 ng/ml (e.g., from 1 ng/mL to 1,000 ng/ml, from 5 ng/mL to 500 ng/mL, from 10 ng/mL to 250 ng/mL, from 50 ng/ml to 150 ng/ml, e.g., from 1 ng/mL to 5 ng/mL, from 5 ng/ml to 10 ng/ml, from 10 ng/ml to 50 ng/ml, from 50 ng/ml to 100 ng/mL, from 100 ng/mL to 150 ng/mL). In certain embodiments, the methods defined herein include IL-7 typically at a concentration of less than 500 ng/mL, such as less than 100 ng/mL. In some embodiments, the methods include IL-7 at a concentration of about 100 ng/mL. In some embodiments, the methods include IL-7 at a concentration of about 10 ng/mL.

As used herein, “IL-21” refers to native or recombinant IL-21 or a variant thereof that acts as an agonist for one or more IL-21 receptor (IL-21R) subunits (e.g., mutants, muteins, analogues, subunits, receptor complexes, fragments, isoforms, and peptidomimetics thereof). Mature human IL-21 occurs as a 133 amino acid sequence (less the signal peptide, consisting of an additional 25 N-terminal amino acids). Alternative splicing generates an additional isoform with a substitution of the C-terminal 16 amino acids.

IL-21 can also refer to IL-21 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-21 variants are also encompassed by the disclosure. Examples of such variants are proteins that result from alternate mRNA splicing events or from proteolytic cleavage of the IL-21 protein, wherein the IL-21 binding property is retained. Alternate splicing of mRNA may yield a truncated but biologically active IL-21 protein. Variations attributable to proteolysis include, for example, differences in the N- or C-termini upon expression in different types of host cells, due to proteolytic removal of one or more terminal amino acids from the IL-21 protein (generally from 1-10 amino acids).

In further embodiments, the methods defined herein include IL-21 typically at a concentration of at least 0.01 IU/mL, such as at least 0.1 IU/mL (e.g., from 0.01 IU/mL to 100 IU/mL, from 0.05 IU/mL to 50 IU/mL, from 0.1 IU/mL to 10 IU/mL, from 1 IU/mL to 5 IU/mL, e.g., from 0.01 IU/mL to 0.05 IU/mL, from 0.05 IU/mL to 0.1 IU/mL, from 0.1 IU/mL to 1 IU/mL, from 5 IU/mL to 10 IU/mL, from 10 IU/mL to 50 IU/mL, from 50 IU/mL to 100 IU/mL). In certain embodiments, the methods defined herein include IL-21 typically at a concentration of less than 10 IU/mL, such as about 5 IU/mL. In some embodiments, the methods include IL-21 at a concentration of about 1 IU/mL, such as 1.05 IU/mL. In further embodiments, the methods include IL-21 at a concentration of 1 to 25 ng/ml. Thus, in one embodiment, the methods include IL-21 at a concentration between 5 ng/ml and 20 ng/mL. In a further embodiment, the methods include IL-21 at a concentration of about 5 ng/ml, for example 5.25 ng/mL.

In some embodiments, the gamma delta T cells are obtained from a human sample. In some aspects, the sample is any sample that contains gamma delta T cells or precursors thereof including, but not limited to, blood, bone marrow, lymphoid tissue, epithelia, thymus, liver, spleen, cancerous tissues, lymph node tissue, infected tissue, fetal tissue, skin, gut, uterus and fractions or enriched portions thereof. In some embodiments, the Vδ1+ T cells are obtained from a blood sample. The sample includes peripheral blood, umbilical cord blood or fractions thereof, including buffy coat cells, leukapheresis products, peripheral blood mononuclear cells (PBMCs) and low density mononuclear cells (LDMCs). In some embodiments, the blood sample is peripheral blood or a fraction thereof. In some embodiments, the sample is human blood or a fraction thereof.

In some embodiments, the sample is non-hematopoeitic tissue. In preferred embodiments, the sample is skin tissue, including dermis, epidermis and gut lining.

The non-hematopoeitic (e.g., skin) sample may be obtained using techniques known in the art such as a biopsy. In some embodiments, cells are separated by density gradient centrifugation. The sample may be fresh or frozen.

### Isolation and Culturing Methods

In some embodiments, the non-hematopoeitic tissues are obtained by methods known in the art, for example, scalpel explant or punch biopsy and vary in size according to the method. In some embodiments, the non-hematopoietic tissue sample is obtained by punch biopsy. In some embodiments, the non-hematopoietic tissue sample is not minced. In another embodiment, the intact tissue is obtained by punch biopsy.

A punch biopsy may be of any shape, though is conveniently of circular cross-section and suitably is at least 1 mm in diameter. In yet further embodiments, the non-hematopoietic tissue sample comprises a punch biopsy at least 2 mm in diameter, such as at least 3 mm in diameter, at least 4 mm in diameter, at least 5 mm in diameter, at least 6 mm in diameter, at least 7 mm in diameter or at least 8 mm in diameter. In further embodiments, the non-hematopoietic tissue sample comprises a punch biopsy 8 mm or less in diameter, such as 7 mm or less in diameter, 6 mm or less in diameter, 5 mm or less in diameter or 3 mm or less in diameter. In some embodiments, the non-hematopoietic tissue sample comprises a punch biopsy of between 1 mm and 8 mm in diameter, such as between 2 mm and 4 mm in diameter. In a particular embodiment, the non-hematopoietic tissue sample comprises a punch biopsy of 3 mm in diameter. In some embodiments, the biopsy is a skin biopsy and comprises the epidermal and dermal layers. In some embodiments, the biopsy does not substantially comprise the subcutaneous fat. In some embodiments, the biopsy comprises epidermal and dermal layers and does not substantially comprise a layer of subcutaneous fat. In some embodiments, the biopsy comprises no subcutaneous fat. Alternatively, the subcutaneous fat is not removed, therefore is present (or at least partially present) in the biopsy. In some embodiments, the biopsy consists of epidermal and dermal layers. In some embodiments, the biopsy comprises the full thickness of the non-hematopoietic tissue sample.

Methods of isolation of gamma delta T cells as defined herein may comprise disruption of the tissue (e.g., mincing) followed by the separation of gamma delta T cells from other cell types. In some embodiments, methods of isolation of gamma delta T cells as defined herein may comprise “crawl-out” of gamma delta T cells and other cell types from an intact non-haematopoietic tissue sample or tissue matrix of the explant or biopsy, wherein the tissue resident lymphocytes physically separate from the tissue matrix without requiring the disruption of the tissue matrix. By maintaining the integrity of the tissue matrix, it has been found that the tissue resident lymphocytes preferentially egress from the tissue matrix with little or no egress of inhibitory cell types such as fibroblasts, which are retained in the explant or biopsy which can then be easily removed at the end of isolation. Thus, in some embodiments, the use of an intact non-haematopoietic tissue sample or tissue matrix leads to a low number of fibroblasts being released from the tissue into the culture. Such “crawl-out” methods utilizing intact non-haematopoietic tissue or tissue matrix have the advantage of reducing the need for excess processing of the non-haematopoietic tissue sample or tissue matrix, maintain the structural integrity of the non-haematopoietic tissue or tissue matrix and provide the unexpected advantage of delivering higher isolated cell yields.

Thus the methods of isolation of non-haematopoietic tissue derived lymphocytes as defined herein include methods for isolating non-haematopoietic tissue derived lymphocytes from an intact biopsy or explant of non-haematopoietic tissue. Such an intact biopsy or explant is one wherein the structural integrity of the biopsy or explant has not been deliberately disrupted within the perimeter of the excision removing the biopsy or explant from the tissue sample. Such an intact biopsy or explant will have the three-dimensional structure largely maintained except for minor disruption caused by handling. This intact biopsy or explant therefore has not been mechanically disrupted, such as by mincing or chopping, nor chemically enzymatically disrupted, for example. An intact biopsy or intact tissue sample may comprise the whole tissue, the complete tissue, a portion of the tissue or all elements of said tissue. For example, in one embodiment the intact biopsy comprises all layers of the skin. In some embodiments, the biopsy comprises the epidermal and dermal layers of the skin. In some embodiments wherein the biopsy is intact, separation and distinction between such layers is maintained. Thus, references herein to “intact” additionally include biopsies of full thickness of the non-hematopoietic tissue sample.

In some embodiments, disrupted tissue is used in the isolation methods of the present invention. In one embodiment, the isolated lymphocyte is an alpha beta T cell. In an alternative embodiment, the isolated lymphocyte is a gamma delta T cell. In a further embodiment, the isolated lymphocyte is a TCR-negative cell (i.e., a cell which is negative for alpha beta TCR and gamma delta TCR expression). TCR negative cells are a good indicator of the presence of natural killer (NK) cells. Therefore, in some embodiments, the isolated lymphocyte is an NK cell. It can be appreciated that more than one type of lymphocyte may be isolated from the same isolation step. Methods of isolation of gamma delta T cells utilizing “crawl-out” or e.g., methods as defined herein, include the culturing of the cells and/or non-hematopoietic tissue sample in the presence of cytokines and/or chemokines sufficient to induce the isolation or separation of gamma delta T cells and/or other lymphocytes as defined herein.

Methods of the present invention comprise culturing non-hematopoietic tissue sample as defined herein. References herein to “culturing” include the addition of cells and/or a non-hematopoietic tissue sample, including isolated, separated, removed, purified or enriched cells from non-hematopoietic tissue sample to media comprising growth factors and/or essential nutrients required and/or preferred by the cells and/or non-hematopoietic tissue sample. It will be appreciated that such culture conditions may be adapted according to the cells or cell population to be isolated from the non-hematopoietic tissue sample according to the invention or may be adapted according to the cells or cell population to be isolated and expanded from the non-hematopoietic tissue sample.

In certain embodiments, culturing of the non-hematopoietic tissue sample is for a duration of time sufficient for the isolation of gamma delta T cells from the non-hematopoietic tissue sample. In alternative embodiments, the culturing of non-hematopoietic tissue sample is for a duration of time sufficient for the isolation of lymphocytes other than gamma delta T cells from the non-hematopoietic tissue sample (e.g., alpha beta T cells and/or NK (natural killer) cells). In certain embodiments, the duration of culture according to the methods defined herein is at least 7 days. In certain embodiments, the duration of culture according to the methods defined herein is at least 14 days. In certain embodiments, the duration of culture according to the methods defined herein is less than 45 days, such as less than 40 days, such as less than 35 days, such as less than 30 days, such as less than 25 days. In a further embodiment, the duration of culture according to the methods defined herein is between 14 days and 35 days, such as between 14 days and 21 days. In a yet further embodiment, the duration of culture according to the methods defined herein is about 19 days, such as 19 days. In another embodiment, the duration of culture according to the methods defined herein is about 21 days, such as 21 days.

In particular embodiments of the present invention, the lymphocytes and/or gamma delta T cells isolated according to methods as defined herein are collected from the culture of non-hematopoietic tissue sample after culturing of the non-hematopoietic tissue sample. Collection of the lymphocytes and/or gamma delta T cells as defined herein may include the physical collection of lymphocytes and/or gamma delta T cells from the culture, isolation of the lymphocytes and/or gamma delta T cells from other lymphocytes (e.g., alpha beta T cells, gamma delta T cells and/or NK cells) or isolation and/or separation of the lymphocytes and/or gamma delta T cells from stromal cells (e.g. fibroblasts). In one embodiment, lymphocytes and/or gamma delta T cells are collected by mechanical means (e.g., pipetting). In a further embodiment, lymphocytes and/or gamma delta T cells are collected by means of magnetic separation and/or labelling. In a yet further embodiment, the lymphocytes and/or gamma delta T cells are collected by flow cytometric techniques such as FACS. Thus, in certain embodiments, the gamma delta T cells are collected by means of specific labelling the gamma delta T cells. In further embodiments, the lymphocytes are collected by means of specific labelling of the lymphocytes to distinguish them from other cells within the culture. It will be appreciated that such collection of lymphocytes and/or gamma delta T cells may include the physical removal from the culture of the non-hematopoietic tissue sample, transfer to a separate culture vessel or to separate or different culture conditions.

It will be appreciated that such collecting of lymphocytes and/or gamma delta T cells is performed after a duration of time sufficient to achieve an isolated population of lymphocytes and/or gamma delta T cells from the non-hematopoietic tissue sample. In certain embodiments, the lymphocytes and/or gamma delta T cells are collected after at least one week, at least 10 days, at least 11 days, at least 12 days, at least 13 days or at least 14 days of culturing of the non-hematopoietic tissue sample. Suitably, the lymphocytes and/or gamma delta T cells are collected after 40 days or less, such as 38 days or less, 36 days or less, 34 days or less, 32 days or less, 30 days or less, 28 days or less, 26 days or less or 24 days or less. In one embodiment, the lymphocytes and/or gamma delta T cells are collected after at least 14 days of culturing of the non-hematopoietic tissue sample. In a further embodiment, the lymphocytes and/or gamma delta T cells are collected after 14 to 21 days of culturing of the non-hematopoietic tissue sample. In a yet further embodiment, the lymphocytes and/or gamma delta T cells are collected after about 19 days of culturing, such as after 19 days. In another embodiment, the lymphocytes and/or gamma delta T cells are collected after about 21 days of culturing, such as after 21 days.

In some embodiments of the present invention, the non-hematopoietic tissue sample is cultured in media which contains serum (e.g., human AB serum or fetal bovine serum (FBS)). In a further embodiment, the non-hematopoietic tissue is cultured in media containing 10% human AB serum. In another embodiment, the non-hematopoietic tissue is cultured in media containing 5% human AB serum. According to this embodiment, a serum replacement as defined herein may additionally be contained in the media. Thus, in a yet further embodiment the non-hematopoietic tissue is cultured in media containing 5% human AB serum and 5% serum replacement. In certain embodiments of the present invention, the non-hematopoietic tissue sample is cultured in media which contains plasma (e.g., human plasma). In a further embodiment, the hematopoietic tissue is cultured in media containing 2.5% human plasma.

In an alternative embodiment of the present invention, the non-hematopoietic tissue sample is cultured in media which is substantially free of serum (e.g., serum-free media or media containing a serum-replacement (SR)). In a further embodiment, the non-hematopoietic tissue is cultured in media containing 5% serum replacement. Thus, in one embodiment, the non-hematopoietic tissue sample is cultured in serum-free media. Such serum free medium may also include serum replacement medium, where the serum replacement is based on chemically defined components to avoid the use of human or animal derived serum.

In some embodiments, the non-haematopoietic tissue sample is cultured in media which contains no animal-derived products.

As described herein, the compositions and methods of the disclosure may be used with allogeneic derived gamma delta T cells, i.e., cells derived from a sample obtained from another donor. In some embodiments, the gamma delta T cells are obtained from a healthy donor.

In some aspects, prior to culturing the sample or fraction thereof, the sample or fraction thereof may be enriched for certain cell types and/or depleted for other cell types. In some embodiments, the sample is enriched for T cells. In some embodiments, the sample is enriched for TCRγδ+ T cells. For example, the sample may be depleted of TCRα+ T cells, depleted of αβ T cells, non-TCRγδ+ T cells and/or enriched for CD3+ cells. In some embodiments, the sample is first depleted of TCRα+ T cells, and then enriched for CD3+ cells.

In some aspects, the sample may be enriched or depleted of certain cell types using techniques known in the art. In some embodiments, the cells of a particular phenotype may be depleted by culturing the sample or fraction thereof with an antibody cocktail containing antibodies that bind to specific molecules on the cells to be depleted. Preferably, the antibodies in the cocktail are coupled to magnetic microbeads that can be used to magnetically deplete or enrich target cells when these cells are forced to pass through a magnetic column. In some embodiments, the sample is depleted of αβ T cells.

In some embodiments, 6 days after isolation of non-hematopoeitic tissue, cells expressing αβ T cell receptor are removed. In some embodiments, the removal of cells expressing αβ T cell receptor is carried out using magnetic-activated cell sorting (MACS). In some embodiments, the MACS is carried out using QuadraMACS or CliniMACS.

Magnetic-activated cell sorting (MACS) is a method for separation of cell populations depending on their cell surface antigens or cluster of differentiation markers (CD markers). In the MACS method, superparamagnetic nanoparticles of the order of 100 nm are used to tag target cells in order to capture them inside a column, for example, magnetic nanoparticles are coated with antibodies against a cell surface antigen. The cells expressing this antigen are then bound to the magnetic nanoparticles. After incubation of beads with cells, the solution is transferred to a column, which is placed between permanent magnets so that when the magnetic particle-cell complex passes through it, the tagged cells, e.g., cells expressing the antigen attached to the nanoparticles, can be captured. Other cells that do not express the antigen flow through. In some embodiments, the column consists of steel wool increasing the magnetic field gradient to maximize separation efficiency when the column is placed between the permanent magnets.

Using MACS, cells can be separated positively or negatively with respect to the particular antigen(s). With positive selection, the cells expressing the antigen(s) of interest, which attached to the magnetic column, are washed out to a separate vessel, after removing the column from the magnetic field. With negative selection, the antibody used is against surface antigen(s) which are known to be present on cells that are not of interest. After administration of the cells/magnetic nanoparticles solution onto the column the cells expressing these antigens bind to the column and the fraction that goes through is collected, as it contains almost no cells with these undesired antigens.

In some embodiments, magnetic nanoparticles coated with anti-fluorochrome antibodies are incubated with the fluorescent-labelled antibodies against the antigen of interest for cell separation based on cell surface antigen.

In some aspects, the collection of the Vδ1+ T cells may include the physical collection of Vδ1+ T cells from the culture, isolation of the Vδ1+ T cells from other lymphocytes (e.g., αβ T cells, γδ T cells and/or NK cells) or isolation and/or separation of the Vδ1+ T cells from stromal cells (e.g., fibroblasts). In some embodiments, Vδ1+ T cells are collected by mechanical means (e.g., pipetting). In some embodiments, Vδ1+ T cells are collected by means of magnetic separation and/or labelling. In some embodiments, the Vδ1+ T cells are collected by flow cytometric techniques such as FACS. Thus, in certain embodiments, the Vδ1+ T cells are collected by means of specific labelling the Vδ1+ T cells. It will be appreciated that such collection of Vδ1+ T cells may include the physical removal from the culture, transfer to a separate culture vessel or to separate or different culture conditions.

In some embodiments, the non-hematopoeitic tissue comprises between 1×10⁹ to 3×10⁹ T cells per 100 cm² surface area.

In some aspects, upon isolation from the sample, the gamma delta T cells will generally be part of a larger population of lymphocytes containing, for example, αβ T cells, B cells, and natural killer (NK) cells. In most cases, the γδ T cell population will include a predominant population of Vδ1 T cells. In some embodiments, 0.1%-10% of the isolated population of lymphocytes are Vδ1+ T cells, e.g., 1-10% of the isolated population of lymphocytes are Vδ1+γδ T cells. In some embodiments, the percentage of Vδ1+ T cells is measured in proportion of CD45+ cells (leukocyte common antigen). In some embodiments, the isolated population is depleted of other cell types (e.g., depleted of αβ T cells). In some embodiments, the isolated population of CD45+ cells depleted of αβ T cells comprises at least 0.1% Vδ1+ T cells, such as at least 0.5% Vδ1+ T cells. In some aspects, once the cells in the sample have been fractionated and enriched, if desired, the cells may be cultured.

### Bioreactor

In some embodiments, the cells are cultured in a GREX100M unit. In some embodiments, the cells are cultured in a serum-free medium formulated for growing T cells comprising glutamine. In some embodiments, the medium is an AIM V medium.

In some embodiments, cells are cultured in a medium formulated for growing T cells comprising human albumin and glutamine but lacking animal-derived components and antibiotics. In some embodiments, the medium is a TexMACS Medium.

In some embodiments, the medium is supplemented with one or more cytokines. In some embodiments, the one or more cytokines is IL-2, IL-15, IL-4 or IL-21. In some embodiments, IL-4 and IL-21 are added once during culturing. In some embodiments, IL-4 and IL-21 are added on the day of isolating non-hematopoeitic (e.g., skin) tissue (D0).

In some embodiments, the one or more cytokines is IL-7, IL-15, IL-4 or IL-21. In some embodiments, IL-4 and IL-7 are added once during culturing. In some embodiments, IL-4 and IL-7 are added on the day of isolating non-hematopoeitic (e.g., skin) tissue (D0). In some embodiments, the one or more cytokines is IL-15 and IL-21. In some embodiments, the one or more cytokines is IL-15 and IL-2. In some embodiments, the one or more cytokines is IL-15, IL-21 and IL-7. In some embodiments, the one or more cytokines is IL-15, IL-21 and IL-4.

The T cells may be expanded in a functionally closed system, such as a bioreactor. Expansion may be performed in a gas-permeable bioreactor, such as GREX cell culture device. The bioreactor may support between 1×10⁹ and 3×10⁹ total cells in an average 450 mL volume.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel).

Hollow fiber bioreactors can be used to enhance the mass transfer during culture. A hollow fiber bioreactor is a 3D cell culturing system based on hollow fibers, which are small, semi-permeable capillary membranes arranged in parallel array with a typical molecular weight cut-off (MWCO) range of 10-30 kDa. These hollow fiber membranes are often bundled and housed within tubular polycarbonate shells to create hollow fiber bioreactor cartridges. Within the cartridges, which are also fitted with inlet and outlet ports, are two compartments: the intracapillary (IC) space within the hollow fibers, and the extracapillary (EC) space surrounding the hollow fibers.

Hollow fiber bioreactors may have the cells embedded within the lumen of the fibers, with the medium perfusing the extralumenal space or, alternatively, may provide gas and medium perfusion through the hollow fibers, with the cells growing within the extralumenal space.

The hollow fibers are suitable for the delivery of nutrients and removal of waste in the bioreactor. The hollow fibers are of any shape, for example, they may be round and tubular or in the form of concentric rings. The hollow fibers may be made up of a resorbable or non-resorbable membrane. For example, suitable components of the hollow fibers include polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, and mixtures thereof.

The bioreactor is primed prior to seeding of the cells. In some embodiments, the priming comprises flushing with a buffer, such as PBS. In some embodiments, the priming comprises coating the bioreactor with an extracellular matrix protein, such as fibronectin. The bioreactor is then washed with media, such as alpha MEM.

In specific embodiments, the present invention uses a GREX bioreactor. The base of the GREX flask is a gas permeable membrane on which cells reside. Hence, cells are in a highly oxygenated environment, allowing them to be grown to high densities. The system scales up easily and requires less frequent culture manipulations. GREX flasks are compatible with standard tissue culture incubators and cellular laboratory equipment, reducing the specialized equipment and investment required to initiate an adoptive cell therapy (ACT) program.

The cells are seeded in the bioreactor at a density of about 100-1,000 cells/cm², such as about 150 cells/cm², about 200 cells/cm², about 250 cells/cm², about 300 cells/cm², such as about 350 cells/cm², such as about 400 cells/cm², such as about 450 cells/cm², such as about 500 cells/cm², such as about 550 cells/cm², such as about 600 cells/cm², such as about 650 cells/cm², such as about 700 cells/cm², such as about 750 cells/cm², such as about 800 cells/cm², such as about 850 cells/cm², such as about 900 cells/cm², such as about 950 cells/cm², or about 1000 cells/cm². Particularly, the cells are seeded at a cell density of about 400-500 cells/cm², such as about 450 cells/cm².

The total number of cells seeded in the bioreactor are about 1.0×10⁶ to about 1.0×10⁵ cells, such as about 1.0×10⁶ to 5.0×10⁶, 5.0×10⁶ to 1.0×10⁷, 1.0×10⁷ to 5.0×10⁷, 5.0×10⁷ to 1.0×10⁵ cells. In some embodiments, the total number of cells seeded in the bioreactor are about 1.0×10⁷ to about 3.0×10⁷, such as about 2.0×10⁷ cells.

The cells are seeded in any suitable cell culture media, many of which are commercially available. Exemplary media include DMEM, RPMI, MEM, Media 199, HAMS and the like. In one embodiment, the media is alpha MEM media, particularly alpha MEM supplemented with L-glutamine. In some embodiments, the media is supplemented with one or more of the following: growth factors, cytokines, hormones, or B27, antibiotics, vitamins and/or small molecule drugs. In some embodiments, the media may be serum-free.

In some embodiments, the cells are incubated at room temperature. In some embodiments, the incubator is humidified and has an atmosphere that is about 5% CO2 and about 1% O2. In some embodiments, the CO₂ concentration ranges from about 1-20%, 2-10%, or 3-5%. In some embodiments, the O₂ concentration ranges from about 1-20%, 2-10%, or 3-5%.

In some embodiments, cells are isolated 14 days (D14), 19 days (D19) or 21 days (D21) after initiation of culturing. In some embodiments, the cells are isolated two weeks after initiation of culturing (D14).

In some embodiments, the isolated cells comprise greater than 1% of γδ cells. In some embodiments, the isolated cells comprise 1% to 5% of γδ cells. In some embodiments, the isolated cells comprise 1% to 10% of γδ cells. In some embodiments, a proportion of γδ cells in the total isolated cells is determined by Fluorescence Activated Cell Sorting (FACS).

In some embodiments, greater than 60% of isolated γδ cells are Vδ1+ T cells. In some embodiments, greater than 70% of isolated γδ cells are Vδ1+ T cells. In some embodiments, greater than 80% of isolated γδ cells are Vδ1+ T cells. In some embodiments, greater than 90% of isolated γδ cells are Vδ1+ T cells. In some embodiments, greater than 95% of isolated γδ cells are Vδ1+ T cells. In some embodiments, greater than 99% of isolated γδ cells are Vδ1+ T cells. In some embodiments, 100% of isolated γδ cells are Vδ1+ T cells.

In some embodiments, the isolated Vδ1+γδ cells have greater than about 80% purity. In some embodiments, the isolated Vδ1+γδ cells have greater than about 90% purity. In some embodiments, the isolated Vδ1+γδ cells have greater than about 95% purity. In some embodiments, the isolated Vδ1+γδ cells have greater than about 99% purity. In some embodiments, the isolated Vδ1+γδ cells have 100% purity.

### Cryoformulation

In some embodiments, the isolated γδ cells are cryopreserved in a cryoformulation medium. Various cryoformulation media for preserving gamma delta T cells are known in the art, as well as methods thereof. In some embodiments, the cryoformulation medium comprises CS10. CryoStor® CS10 is a uniquely formulated cryopreservation medium containing 10% dimethyl sulfoxide (DMSO). TexMACS Medium is an optimized serum-free cell culture medium developed for the cultivation and expansion of T cells, and regulatory T cells. In some embodiments, the cryoformulation medium comprises TexMACS and CS10. In some embodiments, the cryoformulation medium comprises TexMACS and CS10 in a 1:1 ratio.

In some embodiments, suitable freezing solutions contain DMSO and other suitable media supplements, such as human serum albumin, dextran, dextrose, NaCl, Hespan or PlasmaLyte A. Cells then are frozen to a temperature of about −80° C. to about −200° C., such as about −80° C. to about −135° C.

In some embodiments, cryopreservation is accomplished by placing vials in a freezing container and then storing in a −80° C. freezer, for example for 1-3 days, followed by transfer to the vapor phase of a liquid nitrogen storage system. In some embodiments, the isolated gamma delta T cells are frozen in a controlled rate freezer.

In some embodiments, the frozen cells are suitable for long term storage, therefore the isolated cells can remain frozen for a duration of time before subsequent defrosting and expansion. Frozen cells may be stored, for example between −80° C. and −200° C., optionally in liquid nitrogen (vapor phase), until required for use.

In some embodiments, after cryopreservation, cells may be thawed (i.e., defrosted), for example in a 37° C. water bath. The thawed cells may be subsequently used in an expansion method. Methods of expansion may comprise any of the methods described herein, or as described in the art, for example see WO2017072367 and WO2018202808.

Thus, the method may additionally comprise thawing the frozen population of gamma delta T cells. Furthermore, in some embodiments, the method comprises culturing the thawed population of gamma delta T cells for at least 5 days to produce an expanded population of gamma delta T cells.

### Gamma Delta T Cell Compositions

Gamma delta T cells isolated by the methods of the present disclosure express one or more cell surface markers. In some embodiments, the isolated cells express one or more SR01 or SR02 markers.

In some embodiments, the isolated cells express one or more SR01 markers selected from CD27, CD45RA, Programmed cell death protein 1 (PD1), T cell immunoreceptor with Ig and ITIM domains (TIGIT), and killer cell lectin-like receptor subfamily D, member 1 (NKG2D). TIGIT is an immune checkpoint receptor on cytotoxic, memory, and Tregs, as well as NK cells. In some embodiments, the isolated cells express CD27. In some embodiments, the isolated cells express CD45RA. In some embodiments, the isolated cells express Programmed cell death protein 1 (PD1) cells. In some embodiments, the isolated cells express T cell immunoreceptor with Ig and ITIM domains (TIGIT). In some embodiments, the isolated cells express and killer cell lectin-like receptor subfamily D, member 1 (NKG2D).

In some embodiments, the isolated cells express one or more SR02 markers selected from NKp30, CD56, killer cell lectin-like receptor subfamily A, member 1 (NKG2A), and killer cell lectin-like receptor subfamily C, member 1 (NKG2C). In some embodiments, the isolated cells express NKp30. In some embodiments, the isolated cells express CD56. In some embodiments, the isolated cells express killer cell lectin-like receptor subfamily A, member 1 (NKG2A). In some embodiments, the isolated cells express killer cell lectin-like receptor subfamily C, member 1 (NKG2C).

In some embodiments, the isolated cells comprise 60% or greater CD27 cells.

In some embodiments, the isolated cells comprise greater than 85% CD45+ cells. In some embodiments, the isolated cells comprise greater than 90% CD45+ cells. In some embodiments, the isolated cells comprise greater than 95% CD45+ cells. In some embodiments, the isolated cells comprise greater than 99% CD45+ cells. In some embodiments, the isolated cells comprise 100% CD45+ cells.

In some embodiments, the isolated cells comprise less than 80% CD39+ cells.

In some embodiments, the isolated cells comprise greater than 80% CD227+ cells.

In some embodiments, the isolated cells comprise 10% or less Vδ2+ cells.

In some embodiments, the isolated cells have a viability of 70% or greater. In some embodiments, the isolated cells have a viability of 80% or greater. In some embodiments, the isolated cells have a viability of 90% or greater. In some embodiments, the isolated cells have a viability of 95% or greater. In some embodiments, the isolated cells have a viability of 99% or greater. In some embodiments, the isolated cells have a viability of 100%.

In some embodiments, the isolated cells have a yield of between about 1.5×10⁹ to about 2.5×10⁹ cells per 100 cm² surface area. In some embodiments, the isolated cells have a yield of about 1.5×10⁹ cells per 100 cm² surface area. In some embodiments, the isolated cells have a yield of about 2×10⁹ cells per 100 cm² surface area. In some embodiments, the isolated cells have a yield of about 2.5×10⁹ cells per 100 cm² surface area.

In some embodiments, the isolated cells are free of grid impurities. In some embodiments, the isolated cells are free of grid impurities selected from polymers, ceramics, or metals. In some embodiments, the isolated cells are free of impurities selected from tantalum or carbon particulates.

In some embodiments, the cells do not express a chimeric antigen receptor (CAR).

In some embodiments, the cells are transduced to express a chimeric antigen receptor (CAR).

In some embodiments, provided herein is a composition comprising gamma delta (γδ) T cells manufactured by a method described herein.

### Pharmaceutical Compositions and Administration Methods

In some embodiments, provided herein is a method of treating a disease, the method comprising administering a therapeutically effective amount of the composition described herein to a subject in need thereof. In some embodiments, the disease is a cancer, an infectious disease or an inflammatory disease.

Pharmaceutical compositions of the present invention may comprise a target cell population as described herein (e.g., wherein the cell population is obtained or obtainable from any of the methods disclosed herein), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, compositions of the present invention are formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the gamma delta cells described herein may be administered at a dosage of about 10⁴ to 10⁹ cells/kg body weight, for example, about 10⁵ to 10⁶ cells/kg body weight. Gamma delta T cell compositions may also be administered multiple times at these dosages. The cells can be administered by using infusion techniques that are commonly known in immunotherapy. The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i. v. injection. The compositions of T cells may be injected directly into a tumor, lymph node, or site of infection.

In certain embodiments of the present invention, cells activated and expanded using the methods described herein, are administered to a patient in conjunction with (for example, either before, simultaneously or following) any number of relevant treatment modalities, including but not limited to treatment with agents such as antiviral therapy, cidofovir and interleukin-2, cytarabine (also known as ARA-C) or natalizitmab treatment or efaiizumab treatment. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some embodiments, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

Methods of obtaining the Vδ1+ T cells from a sample may comprise additional growth factors. Therefore, in some embodiments, the first or second culture medium, or both culture media, further comprise one or more additional growth factors, e.g., interleukin combinations described herein. In some embodiments, the one or more cytokines is IL-2, IL-15, IL-4 or IL-21. In some embodiments, IL-4 and IL-21 are added once during culturing. In some embodiments, IL-4 and IL-21 are added on the day of isolating skin tissue (D0). In some embodiments, the one or more cytokines is IL-7, IL-15, IL-4 or IL-21. In some embodiments, IL-4 and IL-7 are added once during culturing. In some embodiments, IL-4 and IL-7 are added on the day of isolating skin tissue (D0). In some embodiments, the one or more cytokines is IL-15 and IL-21. In some embodiments, the one or more cytokines is IL-15 and IL-2. In some embodiments, the one or more cytokines is IL-15, IL-21 and IL-7. In some embodiments, the one or more cytokines is IL-15, IL-21 and IL-4.

T cell mitogens refer to any agent that can stimulate T cells through TCR signalling including, but not limited to, plant lectins such as phytohemagglutinin (PHA) and concanavalin A (ConA) and lectins of non-plant origin, antibodies that activate T cells, and other non-lectin/non-antibody mitogens. Preferred antibody clones include anti-CD3 antibodies such as OKT-3 and UCHT-1 clones, anti-γδ antibodies such as B1 and IMMU510, or anti-Vδ1 antibodies. Within the context of the present disclosure, antibodies are understood to include monoclonal antibodies (mAbs), polyclonal antibodies, antibody fragments (e.g., Fab, and F(ab′) 2), single chain antibodies, single chain variable fragments (scFv) and recombinantly produced binding partners. In some embodiments, the antibody is an anti-CD3 monoclonal antibody (mAb). In some embodiments, the antibody is an anti-Vδ1 antibody. Other mitogens include phorbol 12-myristate-13-acetate (TPA) and its related compounds, such as mezerein, or bacterial compounds (e.g., Staphylococcal enterotoxin A (SEA) and Streptococcal protein A). The T cell mitogens may be soluble or immobilized and more than one T cell mitogen may be used in the method.

In some embodiments, the T cell mitogen is an antibody or a fragment thereof. The antibody or fragment thereof may be an anti-CD3 antibody, for example OKT-3. Alternatively, or additionally, the antibody or fragment thereof may be an anti-TCRγδ antibody, such as a pan-γδ TCR antibody or an anti-TCRVδ1 antibody. References herein to “culturing” include the addition of cells to a media comprising growth factors and/or essential nutrients required and/or preferred by the cells and/or non-haematopoietic tissue sample, e.g., skin. Culturing may be by selective expansion, such as by choosing culturing conditions where gamma delta T cells are preferentially expanded over other cell types present in the sample. Alternatively, the expansion conditions are not selective, and culturing may be followed by depletion of non-target cells (e.g., cells other than gamma delta T cells, such as αβ T cells).

In some embodiments, the culturing is performed in the absence of feeder cells.

In some embodiments, the culturing is performed in the absence of substantial stromal cell contact. In some embodiments, the culturing is performed in the absence of substantial fibroblast cell contact.

In some embodiments, the gamma delta T cells are collected after at least 11 days of culturing, such as at least 14 days of culturing. In some embodiments, the duration of culture according to the methods defined herein is at least 14 days. In some embodiments, the duration of culture according to the methods defined herein is less than 45 days, such as less than 30 days, such as less than 25 days. In some embodiments, the duration of culture according to the methods defined herein is between 14 days and 35 days, such as between 14 days and 21 days. In some embodiments, the duration of culture according to the methods defined herein is about 14 days. In some embodiments, the duration of culture according to the methods defined herein is about 21 days.

In some embodiments, the culturing is performed for a duration (e.g. at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g. from 5 days to 40 days, from 7 days to 35 days, from 14 days to 28 days, or about 21 days) in an amount effective to produce an expanded population of gamma delta T cells. In some embodiments, the culturing is for a period of several hours (e.g. about 2, 3, 4, 5, 6, 7, 8, 9, 10, 15, 18, or 21 hours) to about 35 days (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days). In some embodiments, the culturing is for a period of 14 to 21 days.

It will be understood that if two culture media are used, the culturing in each media may occur for different lengths of time. For example, cells are cultured in the first culture medium for a period of time ranging from about 2 days to about 21 days. In some embodiments, cells are cultured from about 3 days to about 14 days. In some embodiments, cells are cultured from about 4 days to 8 days. The cells may be cultured in the second culture medium for a period of time ranging from about 2 days to about 30 days. In some embodiments, cells are cultured from about 5 days to about 21 days. In some embodiments, cells are cultured from about 10 days to 15 days.

In some embodiments, the culturing is performed in a vessel comprising a gas permeable material. Such materials are permeable to gases such as oxygen, carbon dioxide and/or nitrogen to allow gaseous exchange between the contents of the vessel and the surrounding atmosphere. It will be appreciated that references herein to “vessel” include culture dishes, culture plates, single-well dishes, multi-well dishes, multi-well plates, flasks, multi-layer flasks, bottles (such as roller bottles), bioreactors, bags, tubes and the like. Such vessels are known in the art for use in methods involving expansion of non-adherent cells and other lymphocytes. Vessels comprising a gas permeable material have been found to increase the yield of isolated Vδ1+ T cells. Such vessels were also found to preferentially support Vδ1+ T cells and other lymphocytes over fibroblasts and other stromal cells (e.g., epithelial cells), including adherent cell-types. In some embodiments, fibroblasts and/or other stromal cells (e.g., epithelial cells) are absent from cultures performed in vessels comprising a gas permeable material.

Such vessels comprising gas permeable materials may additionally comprise a gas permeable material that is non-porous. Thus, in some embodiments, the gas permeable material in non-porous. In some embodiments, the gas permeable material is a membrane film such as silicone, fluoroethylene polypropylene, polyolefin, or ethylene vinyl acetate copolymer. Furthermore, such vessels may comprise only a portion of gas permeable material, gas permeable membrane film or non-porous gas permeable material. Thus, in some embodiments, the vessel includes a top, a bottom and at least one sidewall, wherein at least part of the said vessel bottom comprises a gas permeable material that is in a substantially horizontal plane when said top is above said bottom. In some embodiments, the vessel includes a top, a bottom, and at least one sidewall, wherein at least a part of said bottom comprises the gas permeable material that is in a horizontal plane when said top is above said bottom. In some embodiments, the vessel includes a top, a bottom and at least one sidewall, wherein the said at least one sidewall comprises a gas permeable material which may be in a vertical plane when said top is above said bottom or may be a horizonal plane when said top is not above said bottom. It will be appreciated that in such embodiments, only a portion of said bottom or said side wall may comprise a gas permeable material. Alternatively, the entire of said bottom or entire of said sidewall may comprise a gas permeable material. In some embodiments, said top of said vessel comprising a gas permeable material may be sealed, for example by utilization of an O-ring. Such embodiments will be appreciated to prevent spillage or reduce evaporation of the vessel contents. In some embodiments, the vessel comprises a liquid sealed container comprising a gas permeable material to allow gas exchange. In some embodiments, said top of said vessel comprising a gas permeable material is in the horizonal plane and above said bottom and is not sealed. In some embodiments, said top is configured to allow gas exchange from the top of the vessel. In some embodiments, said bottom of the gas permeable container is configured to allow gas exchange from the bottom of the vessel. In some embodiments, said vessel comprising a gas permeable material may be a liquid sealed container and further comprise inlet and outlet ports or tubes. In some embodiments, the vessel comprising a gas permeable material includes a top, a bottom and optionally at least one sidewall, wherein at least a part of said top and said bottom comprise a gas permeable material and, if present, at least part of the at least one sidewall comprises a gas permeable material. Example vessels are described in WO2005/035728 and U.S. Pat. No. 9,255,243 which are herein incorporated by reference. These vessels are also commercially available, such as the G-REX® cell culture devices (interchangeably referred to as GREX) provided by Wilson Wolf Manufacturing, such as the G-REX6 well-plate, G-REX24 well-plate and the G-REX10 vessel.

In some embodiments, the sample is cultured in media which is substantially free of serum (e.g., serum-free media or media containing a serum-replacement (SR)). Thus, in some embodiments, the sample is cultured in serum-free media. Such serum free medium may also include serum replacement medium, where the serum replacement is based on chemically defined components to avoid the use of human or animal derived serum. In some embodiments, the sample is cultured in media which contains serum (e.g., human AB serum or fetal bovine serum (FBS)). In some embodiments, the sample is cultured in media which contains serum-replacement. In some embodiments, the sample is cultured in media which contains no animal-derived products. Culturing the sample in serum-free media has the advantage of avoiding issues with filtration, precipitation, contamination and supply of serum. Furthermore, animal derived products are not favored for use in clinical grade manufacturing of human therapeutics.

Numerous basal culture media suitable for use in the proliferation of γδ T cells are available, in particular medium, such as AIM-V, Iscoves medium and RPMI-1640 (Life Technologies). The medium may be supplemented with other media factors as defined herein, such as serum, serum proteins and selective agents, such as antibiotics. For example, in some embodiments, RPMI-1640 medium containing 2 mM glutamine, 10% FBS, 10 mM HEPES, pH 7.2, 1% penicillin-streptomycin, sodium pyruvate (1 mM; Life Technologies), non-essential amino acids (e.g., 100 mM Gly, Ala, Asn, Asp, Glu, Pro and Ser; 1×MEM non-essential amino acids (Life Technologies)), and 10 pl/L β-mercaptoethanol. In some embodiments, AIM-V medium may be supplemented with CTS Immune serum replacement and amphotericin B. Conveniently, cells are cultured at 37° C. in a humidified atmosphere containing 5% C0₂ in a suitable culture medium during isolation and/or expansion. Examples of other ingredients that may be added to the culture media, include, but are not limited to, plasma or serum, purified proteins such as albumin, a lipid source such as low-density lipoprotein (LDL), vitamins, amino acids, steroids and any other supplements supporting or promoting cell growth and/or survival.

The gamma delta T cells obtained according to the described methods can be separated from other cells that may be present in the final culture using techniques known in the art including fluorescence activated cell sorting, immunomagnetic separation, affinity column chromatography, density gradient centrifugation and cellular panning. The obtained gamma delta T cells may be immediately used in the therapeutic, experimental or commercial applications described herein or the cells may be cryopreserved for use at a later date.

Pharmaceutical compositions may include expanded gamma delta T cell compositions as described herein in combination with one or more pharmaceutically or physiologically acceptable carrier, diluents, or excipients. Such compositions may include buffers such as neutral buffered saline or phosphate buffered saline; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. Cryopreservation solutions which may be used in the pharmaceutical compositions of the disclosure include, for example, DMSO. Compositions can be formulated for any suitable administration, e.g., for intravenous administration.

In some embodiments, the pharmaceutical composition is substantially free of, e.g., there are no detectable levels of a contaminant, e.g., of endotoxin or mycoplasma.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

Various aspects of the invention are described in further detail in the following examples. The following examples describe some of the preferred modes of making and practicing the present invention. However, it should be understood that these examples are for illustrative purposes only and are not meant to limit the scope of the invention.

### Example 1. Comparing Functional Characteristics of Gamma Delta T Cells Isolated Using a Three-Dimensional Scaffold or Grid Vs. A Grid-Free Method

This example compares functional characteristics of gamma delta T cells isolated using grid or grid-free methods (e.g., viability, yield, and phenotype of cells obtained, for example, by evaluating the proportion of cells comprising surface markers such as CD27, CD227 and CD39).

Briefly, a skin biopsy was taken by punching out tissue. The explant was transferred to a culture vessel, for example, GEN2 (GREX100M) and cultured in a serum-free medium formulated for growing T cells, e.g., AIM V. Cytokines, for example, IL-2, IL-15, IL-4, IL-7, IL-21 or combinations thereof were added at D0 and periodic intervals thereafter, for example, D7, D14, etc. After a period of culturing, cells were harvested. In some embodiments, harvested cells were cryopreserved in an exemplary cryopreservation medium, for example, TexMACS and CS10.

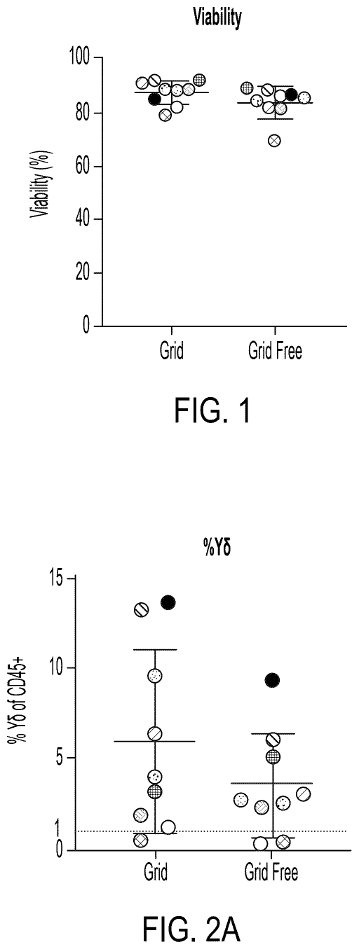

The viability of gamma delta (γδ) cells from grid or grid-free isolation was determined. The results shown in FIG. 1 demonstrated that viability of gamma delta (γδ) cells from grid or grid-free isolation was comparable at about 85%.

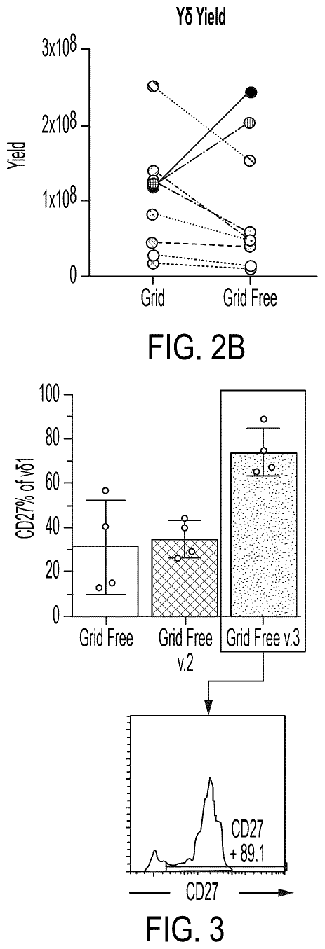

The percentage of gamma delta (γδ) cells from grid or grid-free isolation was measured. The results shown in FIG. 2A demonstrated that the percentage of gamma delta (γδ) cells from grid or grid-free isolation was comparable, though slightly lower in grid-free isolation at about 4% relative to about 6% from grid isolation.

The yield of gamma delta (γδ) cells from grid or grid-free isolation was measured. The results shown in FIG. 2B demonstrated that the γδ yield was slightly lower in grid-free isolation, but most donors met minimum γδ% specification (>1%).

Further, cell phenotypes were evaluated by the presence of cell surface markers. The results shown in FIG. 3 showed the proportion of CD27 cells in three exemplary grid-free cell preparations ranging from 30% to 70%. In version 3, the CD27 percentage was about 70%.

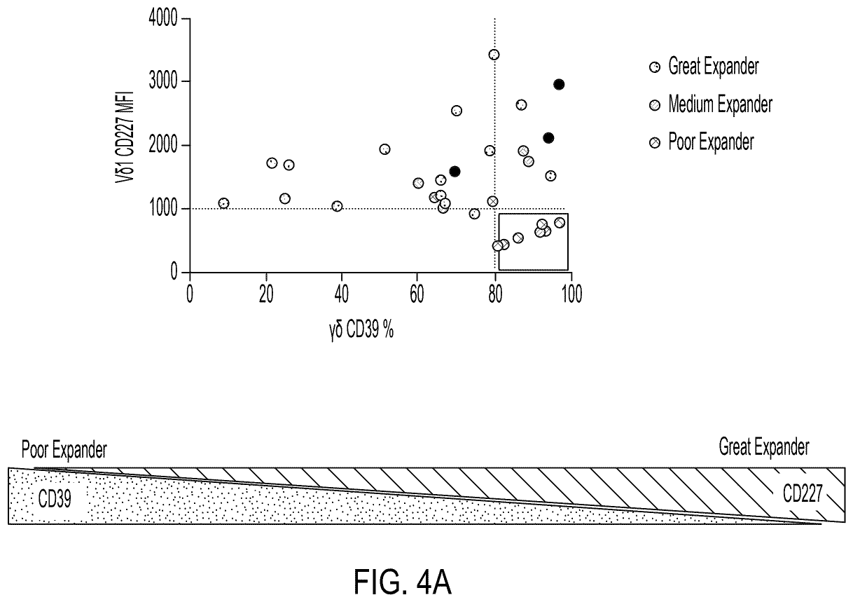

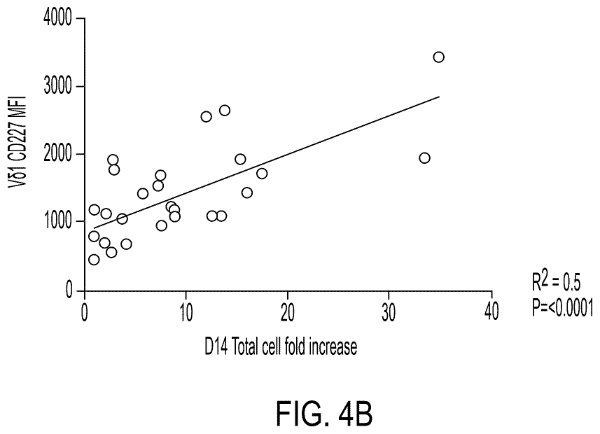

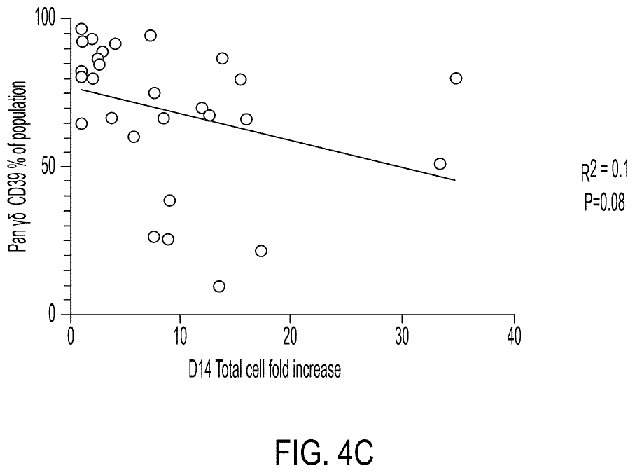

The results shown in FIG. 4A showed the percentage of Vδ1 CD227 vs. percentage of γδ CD39 cells. Based on the graph, high-expanding donors were selected. The results shown in FIG. 4B showed the percentage of total cell fold increase on D14 relative to percentage of Vδ1 CD227 cells. The results shown in FIG. 4C showed the percentage of total cell fold increase on D14 relative to percentage of γδ CD39 cells.

Overall, the results showed comparable viability, total cell yield and extended phenotype, i.e., comparable functional potency, between grid and grid-free isolations.

### Example 2. Testing Cytokine Combinations for Isolating and Expanding Gamma Delta (γδ) T Cells without Use of a Three-Dimensional Scaffold or Grid

This example illustrates a method of isolating and expanding gamma delta T cells from skin tissue without use of a three-dimensional scaffold or grid. Different cytokine combinations were tested for effects on the final yield, viability and phenotype of cells generated.

Briefly, skin biopsy punches were harvested from four donors and seeded in triplicate wells of 10 different formulations comprising different cytokine combinations as depicted in Table 1. The cell culture medium was changed on D7 and D14 after seeding. Triplicate wells were harvested on D19.

TABLE 1
Cytokine combinations

| Condition (C) | Cytokine combinations tested |
|---|---|
| C1 | GEN2 (AIM V media comprising 2.5% allo-plasma and IL-2, |
| | IL-15, IL-4 and IL-1B (D 0 and continuously replenished |
| | throughout the cultureprocess at discrete feeding steps) |
| C2 | IL-2, IL-15 (D 0 IL-4, IL-21) |
| C3 | IL-4, IL-7, IL-15, IL-21 |
| C4 | IL-15 (D 0 IL-4, IL-21) |
| C5 | IL-7, IL-15, IL-21 |
| C6 | IL-15, IL-21 |
| C7 | IL-4, IL-15, IL-21 |
| C8 | IL-15, IL-21 (D 0 IL-4) |
| C9 | IL-7, IL-15, IL-21 (D 0 IL-4) |
| C10 | IL-15, IL-21 (D 0 IL-4, IL-7) |

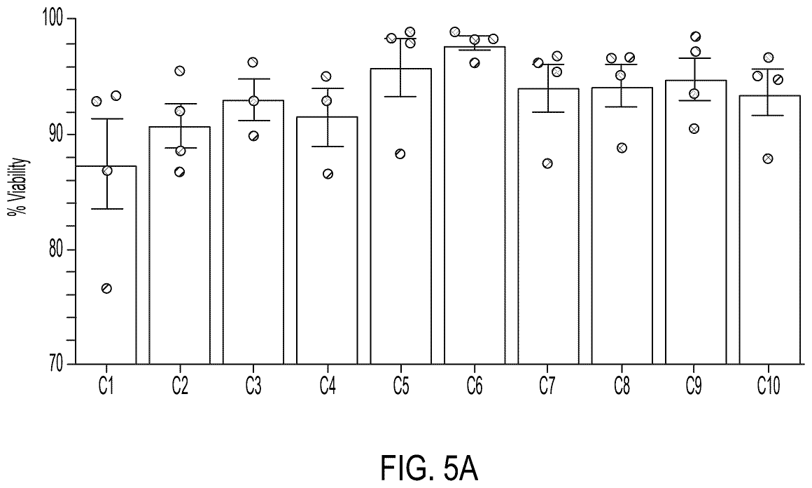

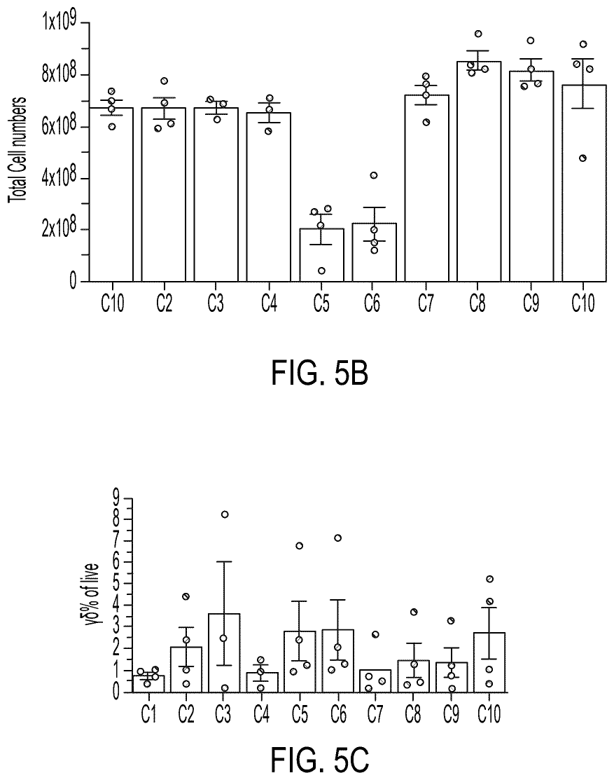

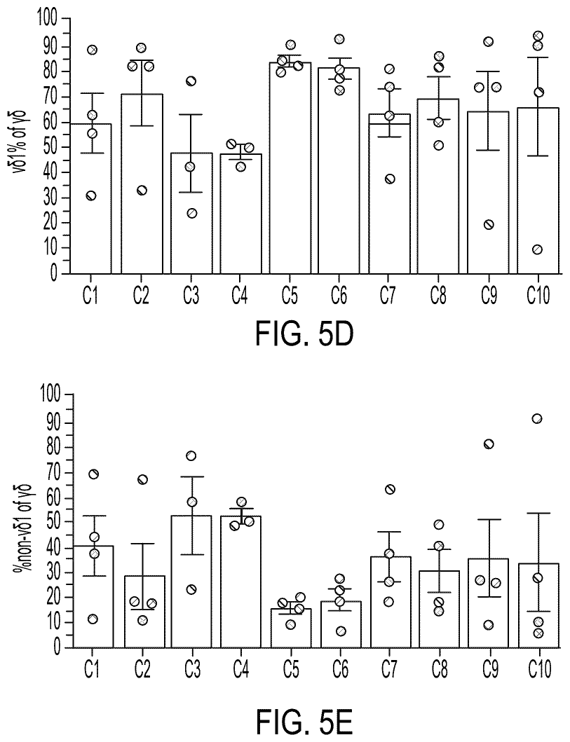

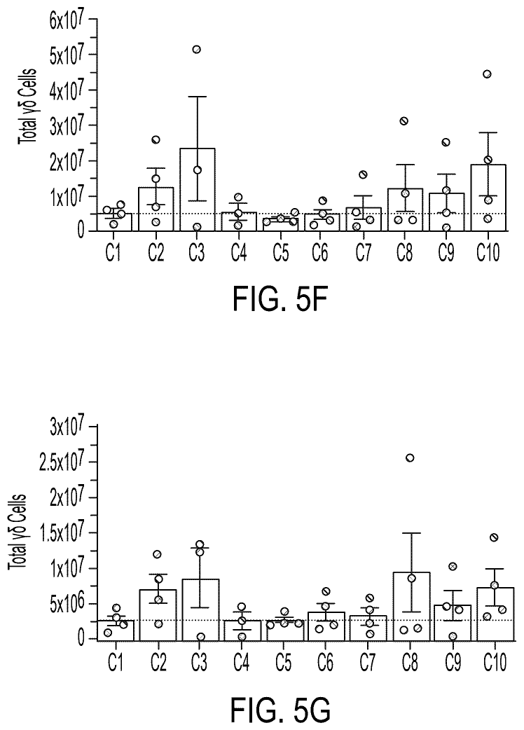

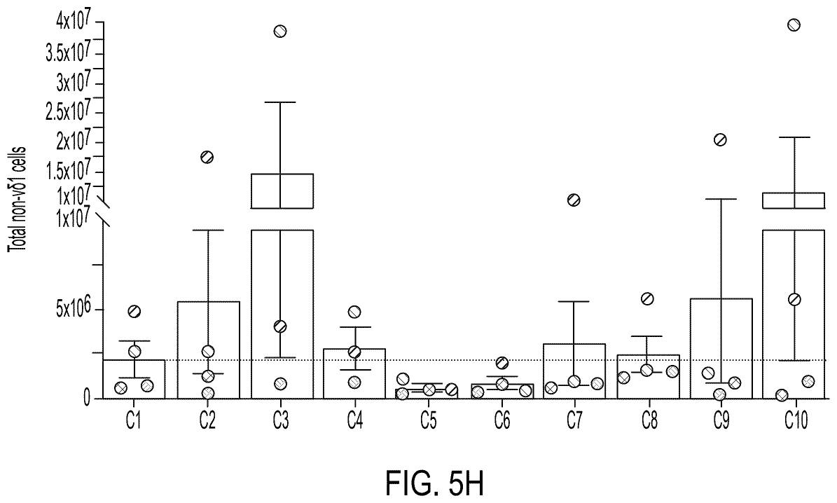

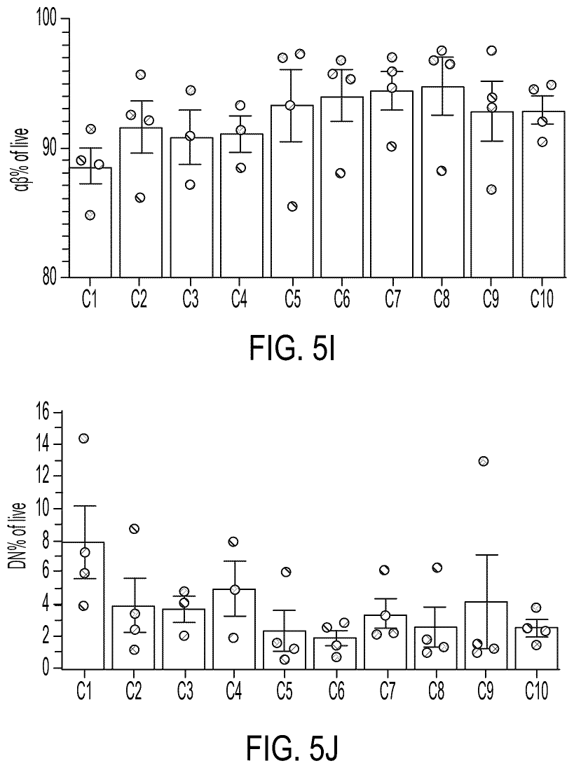

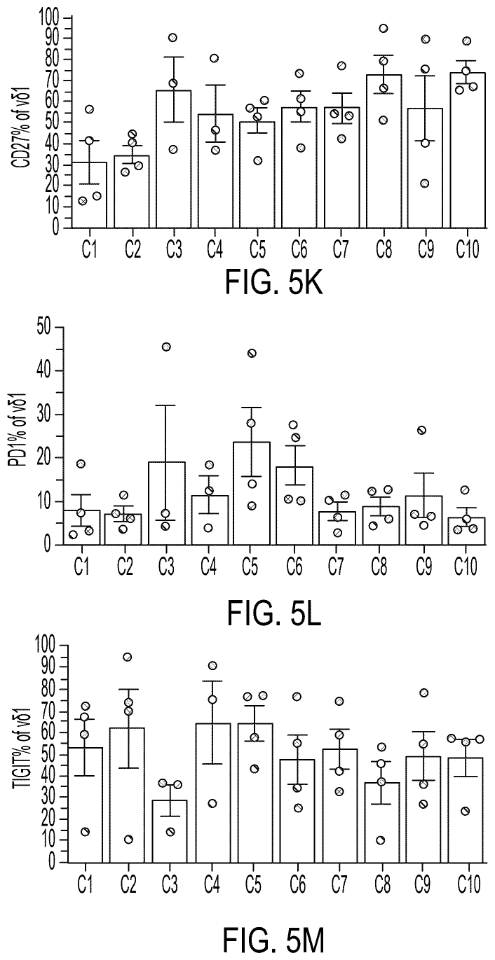

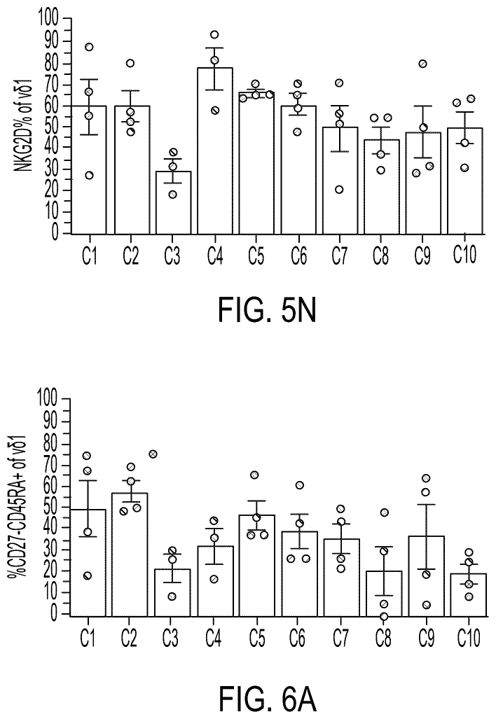

In some embodiments, exemplary concentrations of cytokines used were: 138 IU/mL of IL-2, 600 IU/mL of IL-15, 95 IU/mL of IL-4, 5.25 ng/ml of IL-21 and 10 ng/mL of IL-7 as applicable in the different conditions. In some embodiments, in the GEN2 condition, IL-1B was used at a concentration of 4500 IU/mL. The results shown in FIG. 5A showed that the viability of the cells was comparable between the combinations tested.

The results shown in FIG. 5B showed that yield of the cells was comparable to GEN2 in C1-C4, but decreased significantly in C5 and C6, while yield increased in at least 3 of 4 donors in C7-C10.

The results shown in FIG. 5C showed that although there is donor to donor variation, the percentage of gamma delta (γδ) T cells relative to total live cells was comparable, if not higher between GEN2 and other combinations. The results in FIG. 5D showed that the average percentage of V-delta 1 type of gamma delta T cells was comparable, or higher than GEN2 for C5-C10. The results in FIG. 5E showed that C3 and C4 have a higher percentage on average of non-V-delta 1 type of gamma delta T cells than GEN2.

The results shown in FIG. 5F showed that conditions C2, C3, C7, C8, C9 and C10 showed higher γδ cell number yields than GEN2 with these conditions also having higher numbers of Vδ1 cells (FIG. 5G). Conditions C5 and C6 showed a lower percentage of non-V-delta 1 type cells (FIG. 5H).

The results shown in FIG. 5I showed that the percentage of alpha beta cells was slightly higher than GEN2 in conditions C5-C10 in at least 3 out of 4 donors. The percentage of DN cells was also reduced when compared to GEN2 (FIG. 5J).

The results shown in FIG. 5K showed the percentage of CD27 positive V delta 1 cells. The results showed that CD27 expression was higher than GEN2 in C5-C10. The results shown in FIG. 5L showed the percentage of PD1 positive V delta 1 cells was higher than GEN2 for conditions C3, C5 and C6. The results shown in FIG. 5M showed the percentage of TIGIT positive V delta 1 cells was comparable across conditions except for lower expression for condition C3. The results shown in FIG. 5N showed the percentage of NKG2D positive V delta 1 cells was comparable across conditions except for lower expression for condition C3.

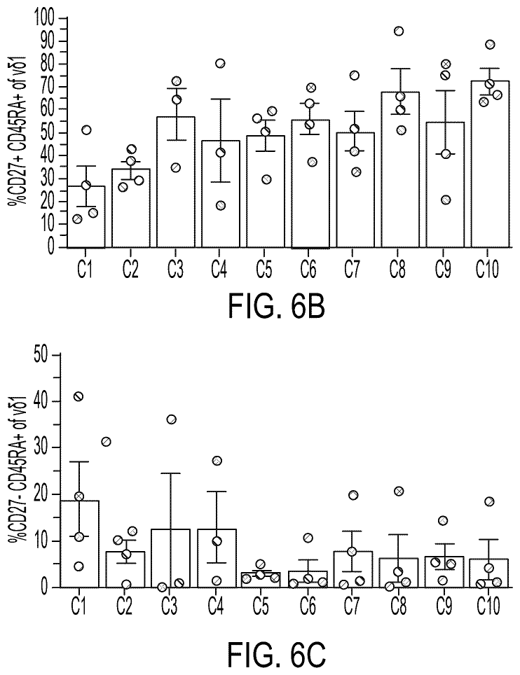

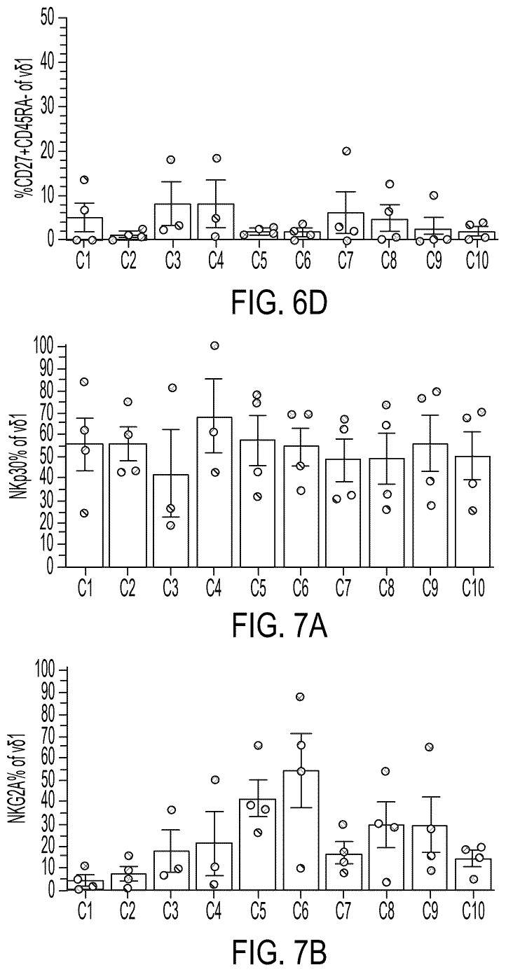

The results shown in FIG. 6A-FIG. 6D showed that conditions 5-10 have enhanced naïve cell phenotypes (CD27 (+) CD45RA (+)) (FIG. 6B) as measured by the presence of both CD27 and CD45R cell surface markers. As shown in FIG. 6B, condition 10 has a strong, consistent, naïve phenotype with lower percentage of differentiating cells in the remaining 3 phenotypes. Other phenotypes tested include terminally differentiated effector memory (CD27(−) CD45RA (+)) (FIG. 6A), effector memory (CD27(−) CD45RA(−)) (FIG. 6C), and central memory (CD27 (+) CD45RA(−)) (FIG. 6D) phenotypes.

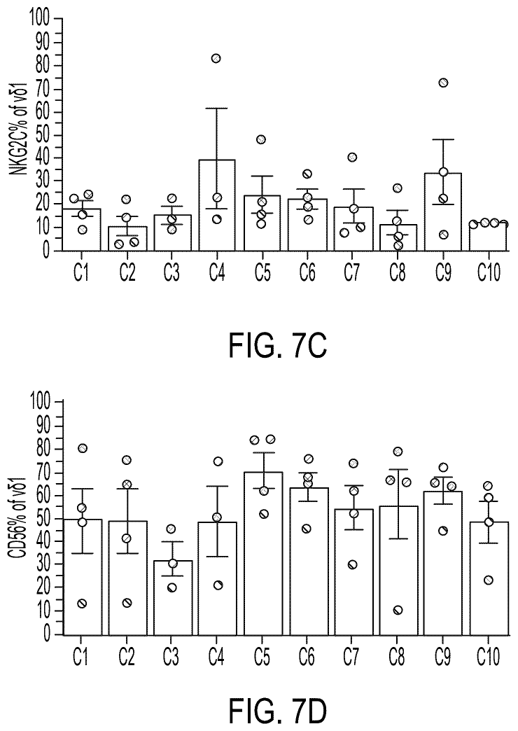

FIG. 7A-FIG. 7D showed SR02 marker expression. As shown in FIG. 7A, NKp30 expression is comparable to GEN2. As shown in FIG. 7B, NKG2A expression increases relative to GEN2 for conditions C5-C10, with the highest increase seen for conditions 5 and 6. As shown in FIG. 7C, NKG2C expression is comparable to GEN2. As shown in FIG. 7D, CD56 expression increases relative to GEN2 for at least 3 of 4 donors for conditions C5-C10.

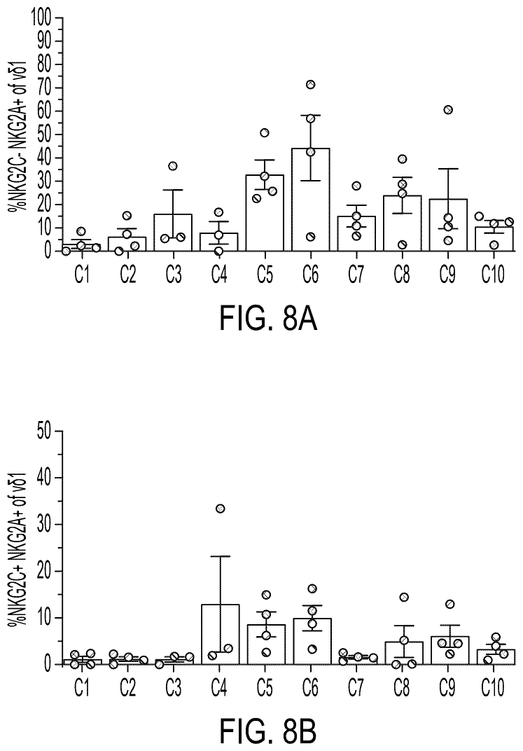

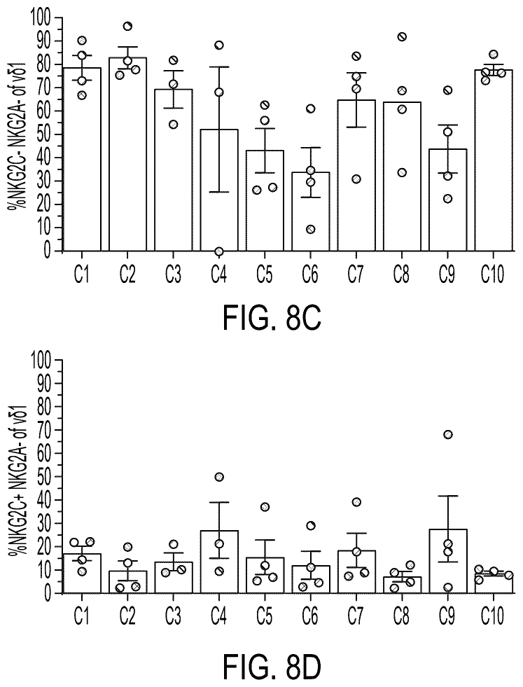

FIG. 8A-FIG. 8D showed NKG2C and NKG2A cell surface markers. FIG. 8A showed NKG2C-NKG2A+ cells. FIG. 8B showed NKG2C+NKG2A+ cells. FIG. 8C showed NKG2C-NKG2A-cells. FIG. 8D showed NKG2C+NKG2A-cells.

As summarized in the results, various cytokine combinations in a grid-free culture showed comparable or improved yield, viability and beneficial phenotypes relative to GEN2 for at least some of the exemplary donors tested.

### Example 3. Methods for Isolating and Expanding Gamma Delta (γδ) T Cells without Use of a Three-Dimensional Scaffold or Grid

This example illustrates a method of isolating and expanding gamma delta T cells from skin tissue without use of a three-dimensional scaffold or grid.

Briefly, DermaPack material from different formulations treated with different cytokine combinations from Example 1, were transferred to T175 flasks seeded at about 4.375e7 cells each and rested for 6 days. On D6, removal of cells expressing αβ T cell receptor was carried out using magnetic-activated cell sorting (MACS), e.g., QuadraMACS.

The αβ depleted T cells were seeded about 1.68e5 cells/well and expanded until D14 with a M/W/F feeding schedule with 2× complete TexMACS supplemented with IL15 (80 ng/ml) and IL21 (11.25 ng/ml). On D14, the αβ-depleted cells were harvested. Staining for SR01 and SR02 markers were carried out to evaluate viability, yield and other parameters.

TABLE 2
Summary of results with different cytokine combinations in exemplary donors tested

| | DermaPack | D 0-D 6 | D 6-D 14 |
|---|---|---|---|
| Viability | C2-C10 have high | Lowest in C1 | C1, C2 and C10 |
| | viability | | comparable |
| Total cell number | C1-C4 comparable | C2, C4, C7 expand | C10 grows 2x of C1 |
| | C8-C10 higher | better in the resting | during the expansion |
| | | phase | phase |
| γδ enrichment | γδ% C10 3x, C2 2x | C1 < C10 < C2 growth. | C10 grew more and |
| | more than C1. | % is comparable | is more γδ pure |
| | γδ cells grow more | | |
| | than C1 | | |
| αβ enrichment | Comparable %, C5- | % comparable | % C2 > C1 > C10 at |
| | C10~2% higher | αβ cell numbers | the end of harvest. |
| | | C2 > C1 > C10 | C10 has the lowest |
| | | | αβ% at the end of |
| | | | expansion |
| Vδ1 enrichment | C2 and C10 higher | C10 has higher | C10 has the highest |
| | in cell numbers than | Vδ1%. Both C2 and | Vδ1 purity (95%), |
| | C1. % vδl higher in | C10 have higher Vδ1 | C2 (60-90%) is |
| | C2 and C10 than C1 | total cells than C1 | higher than C1 (40- |
| | | | 60%). |
| SR01 phenotype | CD27%: All higher | — | CD27%: C10 is |
| | than C1. C10 is | | highest |
| | highest. Naïve | | PD1%: C2-C1, C10 |
| | phenotype is highest | | is lower TIGIT % C2 |
| | for C10. C2 higher | | and C10 > C1 |
| | than C1 | | Naive phenotype |
| | | | higher in C10> |
| | | | C1 > C2 |
| SR02 phenotype | NKG2A is lowest | — | NKG2C is higher in |
| | for C1 | | C10 than C1 and C2 |

Overall, in the exemplary donors tested, while condition 2 (C2; IL-2, IL-15 (D0 IL-4, IL-21)) showed a stronger advantage than GEN2 when the material was generated in a 100M GREX and was αβ-depleted on D0, condition C10 outperformed GEN2 or IL2 IL15 (do IL4 IL21) as the end product had a higher naïve population, had lower αβ outgrowth and had double the γδ fold change with more Vδ1 purity.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method of isolating and expanding gamma delta (γδ) T cells, wherein the method comprises:
(a) isolating non-hematopoietic tissue by biopsy or explant, and
(b) culturing the isolated non-hematopoeitic tissue in the presence of one or more cytokines selected from the group consisting of IL-2, IL-15, IL-4, IL-7, IL-21 and combinations thereof; and
in the absence of a three-dimensional scaffold or grid, thereby expanding and isolating gamma delta (γδ) T cells.

2. The method of claim 1, wherein the gamma delta (γδ) cells are Vdelta1+ (Vδ1+) cells.

3. The method of claim 1, wherein the non-hematopoeitic tissue is treated with one or more cytokines after initiation of culturing.

4. The method of claim 1, wherein the non-hematopoeitic tissue is treated with one or more cytokines weekly for the duration of the culturing.

5. The method of claim 1, wherein the non-hematopoeitic tissue is treated with one or more cytokines at least on the day of isolating non-hematopoeitic tissue (D0), after 7 days of culturing (D7) and after an additional 7 days of culturing (D14).

6. The method of any one of the preceding claims, wherein 6 days after isolation of non-hematopoeitic tissue, cells expressing αβ T cell receptor are removed.

7. The method of any one of the preceding claims, wherein the non-hematopoeitic tissue is skin tissue or gut tissue.

8. The method of claim 7, wherein the removal of cells expressing αβ T cell receptor is carried out using magnetic-activated cell sorting (MACS).

9. The method of claim 8, wherein the MACS is carried out using QuadraMACS or CliniMACS.

10. The method of claim 7, wherein the skin tissue comprises between 1×10⁸ to 2×10¹² T cells per 100 cm² surface area.

11. The method of any one of the preceding claims, wherein the cells are cultured in a GREX100M unit.

12. The method of any one of the preceding claims, wherein the cells are cultured in a serum-free medium formulated for growing T cells comprising glutamine.

13. The method of claim 12, wherein the medium is an AIM V medium.

14. The method of any of claims 1 to 11, wherein cells are cultured in a medium formulated for growing T cells comprising human albumin and glutamine but lacking animal-derived components and antibiotics.

15. The method of claim 14, wherein the medium is a TexMACS Medium.

16. The method of any one of claims 12 to 15, wherein the medium is supplemented with one or more cytokines.

17. The method of claim 16, wherein the one or more cytokines is IL-2, IL-15, IL-4 or IL-21.

18. The method of claim 17, wherein IL-4 and IL-21 are added once during culturing.

19. The method of claim 17, wherein IL-4 and IL-21 are added on the day of isolating non-hematopoietic tissue (D0).

20. The method of claim 16, wherein the one or more cytokines is IL-7, IL-15, IL-4 or IL-21.

21. The method of claim 20, wherein IL-4 and IL-7 are added once during culturing.

22. The method of claim 21, wherein IL-4 and IL-7 are added on the day of isolating non-hematopoeitic tissue (D0).

23. The method of claim 16, wherein the one or more cytokines is IL-15 and IL-21.

24. The method of claim 16, wherein the one or more cytokines is IL-15 and IL-2.

25. The method of claim 16, wherein the one or more cytokines is IL-15, IL-21 and IL-7.

26. The method of claim 16, wherein the one or more cytokines is IL-15, IL-21 and IL-4.

27. The method of any one of the preceding claims, wherein cells are isolated 14 days (D14), 19 days (D19) or 21 days (D21) after initiation of culturing.

28. The method of claim 27, wherein the cells are isolated two weeks after initiation of culturing (D14).

29. The method of any one of the preceding claims, wherein the isolated cells comprise greater than 1% of γδ cells.

30. The method of any one of the preceding claims, wherein the isolated cells comprise 1% to 5% of γδ cells.

31. The method of any one of the preceding claims, wherein the isolated cells comprise 1% to 10% of γδ cells.

32. The method of any one of claims 29 to 31, wherein a proportion of γδ cells in the total isolated cells is determined by Fluorescence Activated Cell Sorting (FACS).

33. The method of claim 32, wherein greater than 60% of isolated γδ cells are Vδ1+ T cells.

34. The method of claim 33, wherein greater than 70% of isolated γδ cells are Vδ1+ T cells.

35. The method of claim 34, wherein greater than 80% of isolated γδ cells are Vδ1+ T cells.

36. The method of claim 35, wherein greater than 90% of isolated γδ cells are Vδ1+ T cells.

37. The method of claim 36, wherein greater than 95% of isolated γδ cells are Vδ1+ T cells.

38. The method of claim 37, wherein greater than 99% of isolated γδ cells are Vδ1+ T cells.

39. The method of claim 38, wherein 100% of isolated γδ cells are Vδ1+ T cells.

40. The method of any one of claims 33 to 39, wherein the isolated Volt γδ cells have greater than about 80% purity.

41. The method of claim 40, wherein the isolated Vδ1+γδ cells have greater than about 90% purity.

42. The method of claim 41, wherein the isolated Vδ1+γδ cells have greater than about 95% purity.

43. The method of claim 42, wherein the isolated Vδ1+γδ cells have greater than about 99% purity.

44. The method of claim 43, wherein the isolated Vδ1+γδ cells have 100% purity.

45. The method of any one of the preceding claims, wherein the isolated Vδ1+γδ cells are cryopreserved in a cryoformulation medium.

46. The method of claim 45, wherein the cryoformulation medium comprises CS10.

47. The method of claim 46, wherein the cryoformulation medium comprises TexMACS and CS10.

48. The method of claim 47, wherein the cryoformulation medium comprises TexMACS and CS10 in a 1:1 ratio.

49. The method of any one of the preceding claims, wherein the isolated cells express one or more SR01 or SR02 markers.

50. The method of claim 49, wherein the isolated cells express one or more SR01 markers selected from CD27, CD45RA, Programmed cell death protein 1 (PD1), T cell immunoreceptor with Ig and ITIM domains (TIGIT), and killer cell lectin-like receptor subfamily D, member 1 (NKG2D).

51. The method of claim 49, wherein the isolated cells express one or more SR02 markers selected from NKp30, CD56, killer cell lectin-like receptor subfamily A, member 1 (NKG2A), and killer cell lectin-like receptor subfamily C, member 1 (NKG2C).

52. The method of any one of the preceding claims, wherein the isolated cells comprise 60% or greater CD27 cells.

53. The method of any one of the preceding claims, wherein the isolated cells comprise greater than 85% CD45+ cells.

54. The method of any one of the preceding claims, wherein the isolated cells comprise greater than 90% CD45+ cells.

55. The method of any one of the preceding claims, wherein the isolated cells comprise greater than 95% CD45+ cells.

56. The method of any one of the preceding claims, wherein the isolated cells comprise greater than 99% CD45+ cells.

57. The method of any one of the preceding claims, wherein the isolated cells comprise 100% CD45+ cells.

58. The method of any one of the preceding claims, wherein the isolated cells comprise less than 80% CD39+ cells.

59. The method of any one of the preceding claims, wherein the isolated cells comprise greater than 80% CD227+ cells.

60. The method of any one of the preceding claims, wherein the isolated cells comprise 10% or less Vδ2+ cells.

61. The method of any one of the preceding claims, wherein the isolated cells have a viability of 70% or greater.

62. The method of any one of the preceding claims, wherein the isolated cells have a viability of 80% or greater.

63. The method of any one of the preceding claims, wherein the isolated cells have a viability of 90% or greater.

64. The method of any one of the preceding claims, wherein the isolated cells have a viability of 95% or greater.

65. The method of any one of the preceding claims, wherein the isolated cells have a viability of 99% or greater.

66. The method of any one of the preceding claims, wherein the isolated cells have a viability of 100%.

67. The method of any one of the preceding claims, wherein the isolated cells have a yield of between about 1.5×10⁹ to about 2.5×10⁹ cells per 100 cm² surface area.

68. The method of any one of the preceding claims, wherein the isolated cells have a yield of about 1.5×10⁹ cells per 100 cm² surface area.

69. The method of any one of the preceding claims, wherein the isolated cells have a yield of about 2×10⁹ cells per 100 cm² surface area.

70. The method of any one of the preceding claims, wherein the isolated cells have a yield of about 2.5×10⁹ cells per 100 cm² surface area.

71. The method of any one of the preceding claims, wherein the isolated cells are free of grid impurities.

72. The method of any one of the preceding claims, wherein the isolated cells are free of grid impurities selected from polymers, ceramics, or metals.

73. The method of any one of the preceding claims, wherein the isolated cells are free of impurities selected from tantalum or carbon particulates.

74. The method of any one of the preceding claims, wherein the cells do not express a chimeric antigen receptor (CAR).

75. The method of any one of claims 1 to 73, wherein the cells are transduced to express a chimeric antigen receptor (CAR).

76. A composition comprising gamma delta (γδ) T cells manufactured by a method comprising any one of the preceding claims.

77. A method of treating a disease, the method comprising administering a therapeutically effective amount of the composition of claim 76 to a subject in need thereof.

78. The method of claim 77, wherein the disease is a cancer, an infectious disease or an inflammatory disease.
